SEQUENCE LISTING (64 sequences)

SEQ ID NO: 1 - protein, 689 aa, synthetic construct
  REGION 1..689: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 2 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 678 aa, synthetic construct
  REGION 1..678: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKGGGSGGGSGGGSGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 4 - protein, 213 aa, synthetic construct
  REGION 1..213: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 5 - protein, 687 aa, synthetic construct
  REGION 1..687: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSDDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSQVQLKQSGPGLVQPSQSLSITCTVSGFSLTNYGVHWVRQSPGKGLEWLGVIWSGGNTDYNTPFTSRLSINKDNSKSQVFFKMNSLQSDDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 6 - protein, 214 aa, synthetic construct
  REGION 1..214: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 687 aa, synthetic construct
  REGION 1..687: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 8 - protein, 214 aa, synthetic construct
  REGION 1..214: note = Synthetic
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 666 aa, synthetic construct
  REGION 1..666: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSDDTAIYYCARALTYYDYEFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKGGGSQVQLKQSGPGLVQPSQSLSITCTVSGFSLTNYGVHWVRQSPGKGLEWLGVIWSGGNTDYNTPFTSRLSINKDNSKSQVFFKMNSLQSDDTAIYYCARALTYYDYEFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 10 - protein, 214 aa, synthetic construct; identical to SEQ ID NO: 6
  REGION 1..214: note = Synthetic

SEQ ID NO: 11 - protein, 671 aa, synthetic construct
  REGION 1..671: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 222 aa, synthetic construct
  REGION 1..222: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSDDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 13 - protein, 688 aa, synthetic construct
  REGION 1..688: note = Synthetic
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 14 - protein, 214 aa, synthetic construct
  REGION 1..214: note = Synthetic
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 671 aa, synthetic construct
  REGION 1..671: note = Synthetic
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 222 aa, synthetic construct
  REGION 1..222: note = Synthetic
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 17 - protein, 671 aa, synthetic construct
  REGION 1..671: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 223 aa, synthetic construct
  REGION 1..223: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 19 - protein, 671 aa, synthetic construct
  REGION 1..671: note = Synthetic
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 222 aa, synthetic construct
  REGION 1..222: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 21 - protein, 687 aa, synthetic construct
  REGION 1..687: note = Synthetic
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 22 - protein, 220 aa, synthetic construct
  REGION 1..220: note = Synthetic
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 683 aa, synthetic construct
  REGION 1..683: note = Synthetic
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 222 aa, synthetic construct
  REGION 1..222: note = Synthetic
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 25 - protein, 120 aa, synthetic construct [sequence omitted]

SEQ ID NO: 26 - protein, 107 aa, synthetic construct [sequence omitted]

SEQ ID NO: 27 - protein, 119 aa, synthetic construct [sequence omitted]

SEQ ID NO: 28 - protein, 107 aa, synthetic construct [sequence omitted]

SEQ ID NO: 29 - protein, 119 aa, synthetic construct
  REGION 1..119: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSDDTAIYYCARALTYYDYEFAYWGQGTLVTVSA

SEQ ID NO: 30 - protein, 107 aa, synthetic construct
  REGION 1..107: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 31 - protein, 119 aa, synthetic construct [sequence omitted]

SEQ ID NO: 32 - protein, 107 aa, synthetic construct [sequence omitted]

SEQ ID NO: 33 - protein, 119 aa, synthetic construct [sequence omitted]

SEQ ID NO: 34 - protein, 113 aa, synthetic construct [sequence omitted]

SEQ ID NO: 35 - protein, 7 aa, synthetic construct
  REGION 1..7: note = Synthetic
GFNIKDT

SEQ ID NO: 36 - protein, 6 aa, synthetic construct
  REGION 1..6: note = Synthetic
YPTNGY

SEQ ID NO: 37 - protein, 11 aa, synthetic construct
  REGION 1..11: note = Synthetic
WGGDGFYAMDY

SEQ ID NO: 38 - protein, 11 aa, synthetic construct
  REGION 1..11: note = Synthetic
RASQDVNTAVA

SEQ ID NO: 39 - protein, 7 aa, synthetic construct
  REGION 1..7: note = Synthetic
SASFLYS

SEQ ID NO: 40 - protein, 9 aa, synthetic construct
  REGION 1..9: note = Synthetic
QQHYTTPPT

SEQ ID NO: 41 - protein, 7 aa, synthetic construct
  REGION 1..7: note = Synthetic
GFTFTDY

SEQ ID NO: 42 - protein, 6 aa, synthetic construct
  REGION 1..6: note = Synthetic
NPNSGG

SEQ ID NO: 43 - protein, 10 aa, synthetic construct
  REGION 1..10: note = Synthetic
NLGPSFYFDY

SEQ ID NO: 44 - protein, 11 aa, synthetic construct
  REGION 1..11: note = Synthetic
KASQDVSIGVA

SEQ ID NO: 45 - protein, 7 aa, synthetic construct
  REGION 1..7: note = Synthetic
SASYRYT

SEQ ID NO: 46 - protein, 9 aa, synthetic construct
  REGION 1..9: note = Synthetic
QQYYIYPYT

SEQ ID NO: 47 - protein, 7 aa, synthetic construct
  REGION 1..7: note = Synthetic
GFSLTNY

SEQ ID NO: 48 - protein, 5 aa, synthetic construct
  REGION 1..5: note = Synthetic
WSGGN

SEQ ID NO: 49 - protein, 11 aa, synthetic construct
  REGION 1..11: note = Synthetic
ALTYYDYEFAY

SEQ ID NO: 50 - protein, 11 aa, synthetic construct
  REGION 1..11: note = Synthetic
RASQSIGTNIH

SEQ ID NO: 51 - protein, 7 aa, synthetic construct
  REGION 1..7: note = Synthetic
YASESIS

SEQ ID NO: 52 - protein, 9 aa, synthetic construct
  REGION 1..9: note = Synthetic
QQNNNWPTT

SEQ ID NO: 53 - protein, 9 aa, synthetic construct
  REGION 1..9: note = Synthetic
GGSVSSGDY

SEQ ID NO: 54 - protein, 5 aa, synthetic construct
  REGION 1..5: note = Synthetic
YYSGN

SEQ ID NO: 55 - protein, 9 aa, synthetic construct
  REGION 1..9: note = Synthetic
DRVTGAFDI

SEQ ID NO: 56 - protein, 11 aa, synthetic construct
  REGION 1..11: note = Synthetic
QASQDISNYLN

SEQ ID NO: 57 - protein, 7 aa, synthetic construct
  REGION 1..7: note = Synthetic
DASNLET

SEQ ID NO: 58 - protein, 9 aa, synthetic construct
  REGION 1..9: note = Synthetic
QHFDHLPLA

SEQ ID NO: 59 - protein, 7 aa, synthetic construct
  REGION 1..7: note = Synthetic
GYTFTSY

SEQ ID NO: 60 - protein, 6 aa, synthetic construct
  REGION 1..6: note = Synthetic
DPSNSD

SEQ ID NO: 61 - protein, 10 aa, synthetic construct
  REGION 1..10: note = Synthetic
YRSYVTPLDY

SEQ ID NO: 62 - protein, 17 aa, synthetic construct
  REGION 1..17: note = Synthetic
KSSQSLLYTSSQKNYLA

SEQ ID NO: 63 - protein, 7 aa, synthetic construct
  REGION 1..7: note = Synthetic
WASTRES

SEQ ID NO: 64 - protein, 9 aa, synthetic construct
  REGION 1..9: note = Synthetic
QQYYAYPWT

(19) United States
(12) Patent Application Publication
(10) Pub. No.: US 2024/0342304 A1
(43) Pub. Date: Oct. 17, 2024

(54) Tetravalent Antibody-Drug Conjugates and Use Thereof

(71) Applicant: Bliss Biopharmaceutical (Hangzhou) Co., Ltd., Hangzhou (Zhejiang) (CN)

(72) Inventors: Yuhong Zhou, Phoenixville, PA (US); Ziping Wei, North Potomac, MD (US)

(21) Appl. No.: 18/600,010

(22) Filed: Mar. 8, 2024

Related U.S. Application Data
(63) Continuation of application No. 17/227,536, filed on Apr. 12, 2021, now Pat. No. 11,951,173.
Application No. 17/227,536 is a continuation of application No. 16/862,042, filed on Apr. 29, 2020, now Pat. No. 10,994,021.
(60) Provisional application No. 63/008,726, filed on Apr. 11, 2020.

(51) Int. Cl. A61K 47/68; A61K 47/60; C07K 16/28; C07K 16/32; C07K 16/46

(52) U.S. Cl. CPC A61K 47/6879; A61K 47/60; A61K 47/68031; A61K 47/68033; A61K 47/6849; C07K 16/2863; C07K 16/32; C07K 16/468; C07K 2317/35

(57) ABSTRACT

An antibody-drug conjugate (ADC) or a pharmaceutically acceptable salt thereof, is provided. The ADC includes a tetravalent monoclonal antibody conjugated to a cytotoxic drug by a chemical linker. The monoclonal antibody includes two long chains and four short chains. Each of the long chains includes a first segment and a second segment, the first segment located proximal to the N-terminus of the long chain, and the second segment located proximal to the C-terminus of the long chain. Each of the first segment and second segment pairs with one of the short chains to form an antigen-binding fragment domain, therefore forming four antigen-binding fragment domains having specificity toward the same antigen. Pharmaceutical composition including the ADC and methods of treating diseases using the compositions are also provided.

### CROSS REFERENCE TO RELATED APPLICATION

This is a continuation of U.S. Nonprovisional application Ser. No. 17/227,536, filed Apr. 12, 2021, which is a continuation of U.S. Nonprovisional application Ser. No. 16/862,042, filed Apr. 29, 2020, which claims priority to U.S. Provisional Application No. 63/008,726, filed Apr. 11, 2020, the disclosure of each of which is incorporated by reference herein in its entirety.

### INCORPORATION OF SEQUENCE LISTING

This application includes a Sequence Listing which is being submitted in XML format via Patent Center, named “BLISS001US_CON2.XML,” which is 90 KB in size and created on Mar. 5, 2024. The contents of the Sequence Listing are incorporated herein by reference in their entirety.

### BACKGROUND

Antibodies are key immune molecules acting against foreign pathogens. The development of monoclonal antibody (mAb) technology resulted in widespread use of monoclonal antibodies in research, diagnosis and treatment of diseases. The therapeutic use of first-generation mAb (mostly monospecific, bivalent mAb) achieved success in the treatment of a variety of diseases, including cancer, autoimmune, and infectious diseases. However, many diseases, such as solid tumors, have been shown to be quite resistant to antibody-based therapies.

In order to increase potency, improve the safety profile and acquire non-natural properties of mAb, new formats of mAb with modified, multivalent properties have been developed. For example, novel trivalent and tetravalent antibodies have been developed to maximize tumor targeting capabilities through enhanced biodistribution and functional affinity. For example, tetravalent antibodies have been generated for use in cancer immunotherapy, where two binding portions can react with target molecules on the cancer cell surface, while two other binding portions react with cytotoxic T cells or natural killer (NK) cells.

Antibody Drug Conjugates (ADCs) are mAbs chemically linked to active drugs, and therefore, have both the specific targeting of mAbs and the cancer-killing ability of cytotoxic drugs. The ability to select specific mAbs-drug combination and advances in the linking the mAbs and drugs provides new possibilities to target cancers while minimizing exposure of healthy tissue. By 2019, a total of seven ADCs have been approved by the FDA, including: ado-trastuzumab emtansine (Kadcyla™), brentuximab vedotin (Adcetris™), inotuzumab ozogamicin (Besponsa™), gemtuzumab ozogamicin (Mylotarg™), polatuzumab vedotin-piiq (Polivy™), Enfortumab vedotin (Padcev™), and Trastuzumab deruxtecan (Enhertu™). All of them involve conjugation of a cytotoxic drug with a bivalent mAb. In addition to the seven ADC drugs that have been approved for marketing, a large number of ADCs are currently under clinical development.

HER2 (Human Epidermal Growth Factor Receptor 2) also known as Neu, ErbB-2, CD340 or p185, is a member of the epidermal growth factor receptor (EGFR/ErbB) family encoded by the ERBB2 gene. An acquired somatic alteration of this gene resulting in amplification and overexpression occurs in approximately 20-25%, 23-80%, 10-30%, and 20% of human breast, bladder, gastric, and lung cancers, respectively. Amplification and overexpression result in an increase in the proliferative stimuli associated with HER2. This increase in proliferative stimuli results in increased tumor growth. Therefore, enhanced or constitutive HER2 activity was believed to be the driving force of malignances in HER2-expressing human tumor cells. Trastuzumab and Pertuzumab, HER2-targeting monoclonal antibodies, were developed by Roche that bound HER2 and inhibited the tyrosine kinase signal pathway by preventing the necessary dimerization of HER2 or heterodimerization with other members of the HER family (EGFR, HER3 and HER4), which in turn slowed the growth of HER2 overexpressing (HER2-positive) cancer cells. To further improve the therapeutic benefit of HER2-targeting antibody therapies, Roche launched Trastuzumab emtansinc (T-DM1), which combines the anti-tumor activity of Trastuzumab with the anti-microtubule agent DM1.

The epidermal growth factor receptor (EGFR, also known as HER1 or c-erbB-1) is a 170 kDa transmembrane glycoprotein and a member of the tyrosine kinase family of cell surface receptors. EGFR is abnormally activated in many epithelial tumors, including those in non-small cell lung cancer, breast cancer, colorectal cancer, head and neck cancers, and prostate cancer. Abnormal activation of EGFR can arise from overexpression of the receptor, gene amplification, activating mutations, overexpression of receptor ligands, and/or loss of regulators of EGFR activity.

Interruption of EGFR signaling, either by blocking EGFR binding sites on the extracellular domain of the receptor or by inhibiting intracellular tyrosine kinase activity, can prevent the growth of EGFR-expressing tumors and improve the patient's condition. Anti-EGFR antibodies exert antitumor effects by binding the receptor at the cell surface to interfere with ligand binding, which leads to the inhibition of its downstream signaling pathway. The chimeric monoclonal antibody C225 (or cetuximab), which contains the murine variable region of mAb225 and a human IgG1 constant region, is available for treatment of irinotecan-refractory colon cancer in parts of the world.

The approved naked antibodies (i.e., cetuximab, nimotuzumab, panitumumab, and necitumumab) for EGFR demonstrate their therapeutic utility in malignancies but are often used in combination with chemotherapy drugs to achieve significant clinical efficacy. Due to the suboptimal activities of naked antibodies, antibody-drug conjugates (ADC) as the next generation therapies that may increase the antitumor activity of an antibody. In EGFR-targeted therapy, there is no commercially approved ADC while several ADCs have entered clinical trials. AVID-100, which is composed of the maytansinoid attached to an anti-EGFR antibody, was developed to treat epithelial tumor patients in phase I/II. ABT-414, which is composed of the monomethyl auristatin F attached to an anti-EGFRvIII antibody via a cleavable linker, has shown significant efficacy against tumors expressing amplified EGFR and EGFRvIII in phase III (van den Bent et al., 2017). All clinical stage EGFR-targeting ADCs have utilized the conventional bivalent anti-EGFR mAbs as targeting antibodies.

### SUMMARY

In one aspect, the present disclosure provides an antibody-drug conjugate (ADC), comprising: a tetravalent monoclonal antibody (or tetrabody) conjugated to a cytotoxic drug by a chemical linker, wherein the monoclonal antibody comprises two long chains and four short chains, wherein each of the long chains comprises a first segment and a second segment, the first segment located proximal to the N-terminus of the long chain, and the second segment located proximal to the C-terminus of the long chain, each of the first segment and second segment paring with one of the short chains to form an antigen-binding fragment domain, therefore forming four antigen-binding fragment domains having specificity toward the same antigen.

In some embodiments of the ADC, the two long chains are identical to each other, and the four short chains are identical to each other. In some embodiments, the two long chains can be different from each other. In some embodiments, the four short chains can be not all identical to each other.

In some embodiments, the first segment and the second segment of at least one, or each, of the long chains have identical sequence. In some other embodiments, the first segment and the second segment of at least one, or each, of the long chains can have different sequences.

In some embodiments of the ADC, each of the long chains comprises a subsequence having the sequence of CH2-CH3 domain of one of a native human IgG1, IgG2, IgG4 antibody isotypes, the subsequence disposed between the first segment and the second segment. In some embodiments of the ADC, each of the long chains comprises a subsequence disposed between the first segment and the second segment, the subsequence comprises the Fc (or CH2-CH3) domain of a human IgG1 isotype. In some embodiments, such Fc domain can include one or more mutations for desired improvements of Fc domain functions.

In some embodiments of the ADC, at least one, or each of, the short chains comprises the sequence of VL-CL (λ) or VL-CL (κ) of a native human IgG antibody isotype.

In some embodiments of the ADC, at least one, or each, of the short chains comprises the sequence of VH-CH1, where CH1 is of a human IgG antibody isotype. In some of such embodiments, the VH can contain one or more mutations.

In some embodiments of the ADC, at least one of the first segment or the second segment of at least one of the long chains comprises a sequence of VL-CL (κ) of a native human IgG1, IgG2, or IgG4 antibody isotype.

In some embodiments of the ADC, the long chains each comprises a sequence selected from the group consisting of SEQ ID NOs: 3, 5, and 9, and the short chains each comprises a sequence selected from the group consisting of SEQ ID NOs: 4, 6, and 10.

In some embodiments, the first segment and second segment of each of the long chains each comprises a sequence selected from the group consisting of SEQ ID NOs: 25, 26, 27, 28, 29, 30, 31, 32, 33, and 34.

In some embodiments, the first segment and second segment of each of the short chains each comprises a sequence selected from the group consisting of SEQ ID NOs: 25, 26, 27, 28, 29, 30, 31, 32, 33, and 34.

In some embodiments, each of the long chains comprises the sequence of SEQ ID NO:25 and each of the short chains comprises the sequence of SEQ ID NO:26. In some embodiments, each of the long chains comprises the sequence of SEQ ID NO:26 and each of the short chains comprises the sequence of SEQ ID NO:25. In some embodiments, each of the long chains comprises the sequence of SEQ ID NO:27 and each of the short chains comprises the sequence of SEQ ID NO:28. In some embodiments, each of the long chains comprises the sequence of SEQ ID NO:28 and each of the short chains comprises the sequence of SEQ ID NO:27. In some embodiments, each of the long chains comprises the sequence of SEQ ID NO:29 and each of the short chains comprises the sequence of SEQ ID NO:30. In some embodiments, each of the long chains comprises the sequence of SEQ ID NO:30 and each of the short chains comprises the sequence of SEQ ID NO:29. In some embodiments, each of the long chains comprises the sequence of SEQ ID NO:31 and each of the short chains comprises the sequence of SEQ ID NO: 32. In some embodiments, each of the long chains comprises the sequence of SEQ ID NO:32 and each of the short chains comprises the sequence of SEQ ID NO:31. In some embodiments, each of the long chains comprises the sequence of SEQ ID NO:33 and each of the short chains comprises the sequence of SEQ ID NO:34. In some embodiments, each of the long chains comprises the sequence of SEQ ID NO:34 and each of the short chains comprises the sequence of SEQ ID NO:34.

In some embodiments of the ADC, each of the short chains comprises a sequence selected from the group consisting of SEQ ID NO:2, SEQ ID NO:4, SEQ ID NO:6, SEQ ID NO: 8, SEQ ID NO:10, SEQ ID NO: 12, SEQ ID NO: 14, SEQ ID NO: 16, SEQ ID NO: 18, SEQ ID NO: 20, SEQ ID NO:22, and SEQ ID NO:24.

In some embodiments of the ADC, each of the long chains comprises a sequence selected from the group consisting of SEQ ID NO: 1, SEQ ID NO:3, SEQ ID NO:5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO:21, and SEQ ID NO:23.

In some embodiments of the ADC, the long chains each comprise a sequence of SEQ ID NO: 1 and the short chains each comprise a sequence of SEQ ID NO:2. In some embodiments of the ADC, the long chains each comprise a of SEQ ID NO:3 and the short chains each comprise a sequence of SEQ ID NO:4. In some embodiments of the ADC, the long chains each comprise a sequence of SEQ ID NO:5 and the short chains each comprise a sequence of SEQ ID NO:6. In some embodiments of the ADC, the long chains each comprise a sequence of SEQ ID NO:7 and the short chains each comprise a sequence of SEQ ID NO:8. In some embodiments of the ADC, the long chains each comprise a sequence of SEQ ID NO:9 and the short chains each comprise a sequence of SEQ ID NO:10.

In some embodiments of the ADC, the long chains each comprise a sequence of SEQ ID NO: 11 and the short chains each comprise a sequence of SEQ ID NO: 12. In some embodiments of the ADC, the long chains each comprise a of SEQ ID NO:13 and the short chains each comprise a sequence of SEQ ID NO:14. In some embodiments of the ADC, the long chains each comprise a sequence of SEQ ID NO: 15 and the short chains each comprise a sequence of SEQ ID NO: 16. In some embodiments of the ADC, the long chains each comprise a sequence of SEQ ID NO: 17 and the short chains each comprise a sequence of SEQ ID NO:18. In some embodiments of the ADC, the long chains each comprise a sequence of SEQ ID NO: 19 and the short chains each comprise a sequence of SEQ ID NO:20. In some embodiments of the ADC, the long chains each comprise a sequence of SEQ ID NO:21 and the short chains each comprise a sequence of SEQ ID NO:22. In some embodiments of the ADC, the long chains each comprise a sequence of SEQ ID NO:23 and the short chains each comprise a sequence of SEQ ID NO:24.

In some embodiments, the present disclosure provides an ADC which comprises a monoclonal antibody conjugated to a cytotoxic drug by a chemical linker, wherein the monoclonal antibody comprises two identical long chains and four identical short chains, wherein:
(a) each of the long chains comprises the sequence of SEQ ID NO: 1 and each of the short chains comprises the sequence of SEQ ID NO:2; or
(b) each of the long chains comprises the sequence of SEQ ID NO:3 and each of the short chains comprises the sequence of SEQ ID NO:4; or
(c) each of the long chains comprises the sequence of SEQ ID NO:5 and each of the short chains comprises the sequence of SEQ ID NO:6; or
(d) each of the long chains comprises the sequence of SEQ ID NO:7 and each of the short chains comprises the sequence of SEQ ID NO:8; or
(e) each of the long chains comprises the sequence of SEQ ID NO:9 and each of the short chains comprises the sequence of SEQ ID NO: 10; or
(f) each of the long chains comprises the sequence of SEQ ID NO:11 and each of the short chains comprises the sequence of SEQ ID NO: 12; or
(g) each of the long chains comprises the sequence of SEQ ID NO: 13 and each of the short chains comprises the sequence of SEQ ID NO:14; or
(h) each of the long chains comprises the sequence of SEQ ID NO:15 and each of the short chains comprises the sequence of SEQ ID NO:16; or
(i) each of the long chains comprises the sequence of SEQ ID NO: 17 and each of the short chains comprises the sequence of SEQ ID NO:18; or
(j) each of the long chains comprises the sequence of SEQ ID NO: 19 and each of the short chains comprises the sequence of SEQ ID NO:20; or
(k) each of the long chains comprises the sequence of SEQ ID NO:21 and each of the short chains comprises the sequence of SEQ ID NO:22; or
(l) each of the long chains comprises the sequence of SEQ ID NO:23 and each of the short chains comprises the sequence of SEQ ID NO:24.

In some embodiments of the ADC, the second segment is connected to the remainder of the long chain via a flexible peptide linker comprising an amino sequence of (GₓS)_{y}, where x is a whole number between 1 and 6 inclusive and y is a whole number between 1 and 8 inclusive.

In some embodiments, the antigen is EGFR, and the ADC contains four antigen-binding fragment domains all specifically bind to EGFR. In some embodiments, the antigen is HER2, and the ADC contains four antigen-binding fragment domains all specifically bind to HER2. In some embodiments, the antigen is c-MET, and the ADC contains four antigen-binding fragment domains all specifically bind to c-MET.

In some embodiments of the ADC, the cytotoxic drug is selected from the group consisting of monomethyl auristatin E (MMAE), monomethyl auristatin F (MMAF), auristatin E, auristatin F, maytansine DM1 and DM4, maytansinol, sandramycin, pyrrolobenzodiazepine dimer, anthracyclines, calicheamicin, dolastatin 10, duocarmycin, doxorubicin, thailanstatin A, uncialamycin, amanitins, ricin, diphtheria toxin, eribulin, ¹³¹I, interleukins, tumor necrosis factors, chemokines, and nanoparticles.

In some embodiments of the ADC, the linker is cleavable or non-cleavable, and can be selected from the group consisting of 6-maleimidocaproyl (MC), maleimidopropionyl (MP), valine-citrulline (val-cit), alanine-phenylalanine (ala-phe), p-aminobenzyloxycarbonyl (PAB), N-succinimidyl 4-(2-pyridylthio) valerate (SPP), N-succinimidyl 4-(N-maleimidomethyl)-cyclohexane-1-carboxylate (SMCC), N-succinimidyl (4-iodo-acetyl) aminobenzoate (SIAB), 6-maleimidocaproyl-valine-citrulline-p-aminobenzyloxycarbonyl (MC-vc-PAB), Val-Cit-PABC, N-succinimidyl-4-(2-pyridyldithio) butanoate (SPDB), N-succinimidyl 3-(pyridin-2-yldithio)-propionate (SPDP), Phe-Lys (Fmoc)-PAB, Aloc-D-Ala-Phe-Lys (Aloc)-PAB-PNP, Boc-Phe-(Alloc) Lys-PAB-PNP, and perfluorophenyl 3-(pyridine-2-yldisulfanyl) propanoate.

In some embodiments of the ADC, the drug-linker can be attached to tetravalent antibody through cysteine coupling, amine coupling, terminal coupling, enzymatic coupling, or carbohydrate coupling.

In some embodiments of the ADC, the drug-to-antibody ratio is between 1 and 12 inclusive. In some embodiments, the drug-to-antibody ratio is about 3 or 4.

The present disclosure also provides a pharmaceutically acceptable salt thereof, of the various embodiments of the ADCs as described herein.

In a further aspect, the present disclosure provides a pharmaceutical composition comprising the ADC, or the pharmaceutically acceptable salt thereof, and a pharmaceutically acceptable carrier.

In a further aspect, the present disclosure provides a method of treating a proliferative disease, disorder, or condition, by administering to a subject an effective amount of the pharmaceutical composition described herein. The proliferative disease may be cancers, such as colon cancer, rectal cancer, lung cancer, breast cancer, ovarian cancer, cervical cancer, bladder cancer, esophageal cancer, sarcoma, and head and neck cancer.

### BRIEF DESCRIPTION OF THE DRAWINGS

FIG. 1 is a schematic depiction of the structure of a tetravalent ADC in accordance with embodiments of the present disclosure.

FIG. 2 is a comparison diagram of HIC profiles of certain tetravalent ADCs of some embodiments of the present disclosure and control ADC. Panel A: Ctl-MMAE; Panel B: BB01E-MMAE with low DAR; Panel C: BB01E-MMAE with high DAR.

FIG. 3 shows ELISA binding results of tetravalent antibodies of some embodiments of the present disclosure and control antibody. Panel A: EGFR-binding results; Panel B: HER2-binding results.

FIG. 4 shows FACS analysis of tetravalent anti-EGFR antibodies of some embodiments of the present disclosure and control antibody. Panel A: EGFR high-expressing MDA-MB-468 cells; Panel B: EGFR low-expressing NUGC3 cells.

FIG. 5 shows internalization analysis of tetravalent anti-EGFR antibodies of some embodiments of the present disclosure and control antibody. Panel A: EGFR high-expressing MDA-MB-468 cells; Panel B: EGFR low-expressing NUGC3 cells.

FIGS. 6A-6C show internalization analysis of tetravalent ADCs of some embodiments of the present disclosure and control ADC. 6A: ADC with MMAE payload on EGFR high-expressing MDA-MB-468 cells; 6B: ADC with DM1 payload on MDA-MB-468 cells; 6C: ADC with MMAE payload on EGFR low-expressing NUGC3 cells.

FIGS. 7A-7C show cytotoxicity of tetravalent ADCs of embodiments of the present disclosure and control ADC. 7A: ADC with MMAE payload to EGFR high-expressing MDA-MB-468 cells; 7B: ADC with DM1 payload to MDA-MB-468 cells; 7C: ADC with MMAE payload to EGFR low-expressing NUGC3 cells.

### DETAILED DESCRIPTION

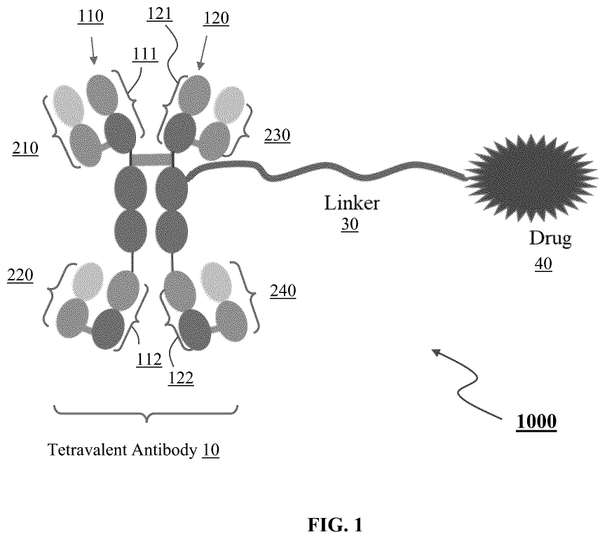

In one aspect, and in connection with illustration shown in FIG. 1, the present disclosure provides an antibody-drug conjugate (ADC) 1000, comprising: a tetravalent monoclonal antibody 10 conjugated to a cytotoxic drug 40 by a chemical linker 30, wherein the monoclonal antibody 10 comprises two long chains (110 and 120) and four short chains (210, 220, 230 and 240), wherein each of the long chains comprises an first segment (111, 121) and a second segment (112, 122), the first segment located proximal to the N-terminus of the long chain, and the second segment located proximal to the C-terminus of the long chain, each of the first segment and second segment paring with one of the short chains to form an antigen-binding fragment domain, therefore forming four antigen-binding fragment domains having binding specificity toward a same antigen.

As used herein, an antibody or a portion thereof that “specifically binds” targets (or target antigen, e.g., HER2, HER3, EGFR, CLDN18.2, TROP2, BMCA, CD33, CD22, CD72 or other antigens) can refer to an antibody or a portion thereof that binds to a human target protein to therefore inhibit its associated signaling. Preferably, the antibody binds to a human target protein with “high affinity,” namely with a KD of 1×10⁻⁸ M or less, more preferably 5×10⁻⁹ M or less or even more preferably 1×10⁻⁹ M or less.

As used herein, the term “cytotoxic drug” or “cytotoxic agent”, as used herein, is meant to refer to any agent or drug known to inhibit the growth and/or replication of, and/or kill cells. As a component of the ADC, it is also referred to as the “payload”.

In some embodiments, the construction of a tetravalent monospecific antibody of the present disclosure can be considered as attaching of the intact Fab domain of a precursor antibody to the Fc C-terminal of the same precursor antibody, or to the Fc C-terminal of a different precursor antibody with the same antigen specificity.

Proper arrangement and spacing of the antigen-binding regions of a tetravalent antibody facilitate multivalent engagement of several molecules of the same antigen on cell surface. Ill-designed antibodies could cause hindrance of some binding epitopes. The design of the invention spaces out the four Fab domains, two on each side of the Fc domain, with the ones on C-terminal linked with a flexible GS linker. This arrangement offers suitable flexibility and ranges for each arm (Fab) to reach (bind to) a target antigen, and can minimize the steric hindrance of different Fabs.

The tetravalent antibodies of the present disclosure could engage target through both N-terminal Fabs and C-terminal Fabs simultaneously, and the multivalent engagement could induce more effective internalization of the target-antibody complex. This offers a distinct advantage of the tetravalent ADC over the traditional ADC.

Multivalent antibodies with scFv fused to the C-terminal of Fc have been reported. The major challenge of such design is the reduced affinities when Fab structure is converted to scFv. Most Mab sequences when converted to scFv became either unstable or with reduced affinity. In contrast, Fab with intact VH-CH1 and VL-CL is generally stable and retains its affinity.

Tetravalent antibody-drug conjugate of the present disclosure takes advantage of the enhanced internalization capability of ADC-target complex through multivalent engagement of antigens on cancer cells that potentially improves the delivery of cytotoxic drugs into target expressing tumor cells.

In some embodiments of the ADC, the two long chains are identical to each other, and the four short chains are identical to each other. In some embodiments, the two long chains can be different from each other. In some embodiments, the four short chains can be not all identical to each other.

In some embodiments of the ADC, the first segment and the second segment of at least one, or each, of the long chains have identical sequence. In some other embodiments, the first segment and the second segment of at least one, or each, of the long chains can have different sequences.

In some embodiments of the ADC, each of the long chains comprises a subsequence having the sequence of CH2-CH3 domain of one of a native human IgG1, IgG2, IgG4 antibody isotypes, the subsequence disposed between the first segment and the second segment.

In some embodiments of the ADC, each of the long chains comprises a subsequence disposed between the first segment and the second segment, the subsequence comprises the CH2-CH3 (or Fc) domain of a human IgG isotype, where such Fc domain can contain one or more mutations for desired improvement of Fc functions.

In some embodiments of the ADC, at least one, or each of, the short chains comprise the sequence of VL-CL (λ) or VL-CL (κ) of a native human IgG antibody isotype.

In some embodiments of the ADC, at least one, or each, of the short chains comprises the sequence of VH-CH1, where CH1 is of a human IgG antibody isotype. The VH portion of the sequence can include one or more mutations.

In some embodiments of the ADC, at least one of the first segment or the second segment of at least one of the long chains comprises a sequence of VL-CL (κ) of a native human IgG1, IgG2, or IgG4 antibody isotype.

In some embodiments of the ADC, the long chains each comprises a sequence selected from the group consisting of SEQ ID NOs: 3, 5, and 9, and the short chains each comprises a sequence selected from the group consisting of SEQ ID NOs: 4, 6, and 10.

In some embodiments of the ADC, the first segment and second segment of each of the long chains each comprises a sequence selected from the group consisting of SEQ ID NOs: 25, 26, 27, 28, 29, 30, 31, 32, 33, and 34.

In some embodiments of the ADC, the first segment and second segment of each of the long chains each comprises a sequence selected from the group consisting of:
(1) an antigen-binding sequence comprising (1a) CDR1 comprising SEQ ID NO:35, (1b) CDR2 comprising SEQ ID NO:36, and (1c) CDR3 comprising SEQ ID NO:37;
(2) an antigen-binding sequence comprising (2a) CDR1 comprising SEQ ID NO:38, (2b) CDR2 comprising SEQ ID NO:39, and (2c) CDR3 comprising SEQ ID NO:40;
(3) an antigen-binding sequence comprising (3a) CDR1 comprising SEQ ID NO:41, (3b) CDR2 comprising SEQ ID NO:42, and (3c) CDR3 comprising SEQ ID NO:43;
(4) an antigen-binding sequence comprising (4a) CDR1 comprising SEQ ID NO:44, (4b) CDR2 comprising SEQ ID NO:45, and (4c) CDR3 comprising SEQ ID NO:46;
(5) an antigen-binding sequence comprising (5a) CDR1 comprising SEQ ID NO:47, (5b) CDR2 comprising SEQ ID NO:48, and (5c) CDR3 comprising SEQ ID NO:49;
(6) an antigen-binding sequence comprising (6a) CDR1 comprising SEQ ID NO:50, (6b) CDR2 comprising SEQ ID NO:51, and (6c) CDR3 comprising SEQ ID NO:52;
(7) an antigen-binding sequence comprising (7a) CDR1 comprising SEQ ID NO:53, (7b) CDR2 comprising SEQ ID NO:54, and (7c) CDR3 comprising SEQ ID NO:55;
(8) an antigen-binding sequence comprising (8a) CDR1 comprising SEQ ID NO:56, (8b) CDR2 comprising SEQ ID NO:57, and (8c) CDR3 comprising SEQ ID NO:58;
(9) an antigen-binding sequence comprising (9a) CDR1 comprising SEQ ID NO:59, (9b) CDR2 comprising SEQ ID NO:60, and (9c) CDR3 comprising SEQ ID NO:61; and
(10) an antigen-binding sequence comprising (10a) CDR1 comprising SEQ ID NO:62, (10b) CDR2 comprising SEQ ID NO:63; and (10c) CDR3 comprising SEQ ID NO: 64.

In some embodiments of the ADC, the first segment and second segment of each of the short chains each comprises a sequence selected from the group consisting of SEQ ID NOs: 25, 26, 27, 28, 29, 30, 31, 32, 33, and 34.

In some embodiments of the ADC, the first segment and second segment of each of the short chains each comprises a sequence selected from the group consisting of:
(1) an antigen-binding sequence comprising (1a) CDR1 comprising SEQ ID NO:35, (1b) CDR2 comprising SEQ ID NO:36, and (1c) CDR3 comprising SEQ ID NO:37;
(2) an antigen-binding sequence comprising (2a) CDR1 comprising SEQ ID NO:38, (2b) CDR2 comprising SEQ ID NO:39, and (2c) CDR3 comprising SEQ ID NO:40;
(3) an antigen-binding sequence comprising (3a) CDR1 comprising SEQ ID NO:41, (3b) CDR2 comprising SEQ ID NO:42, and (3c) CDR3 comprising SEQ ID NO:43;
(4) an antigen-binding sequence comprising (4a) CDR1 comprising SEQ ID NO:44, (4b) CDR2 comprising SEQ ID NO:45, and (4c) CDR3 comprising SEQ ID NO:46;
(5) an antigen-binding sequence comprising (5a) CDR1 comprising SEQ ID NO:47, (5b) CDR2 comprising SEQ ID NO:48, and (5c) CDR3 comprising SEQ ID NO:49;
(6) an antigen-binding sequence comprising (6a) CDR1 comprising SEQ ID NO:50, (6b) CDR2 comprising SEQ ID NO:51, and (6c) CDR3 comprising SEQ ID NO:52;
(7) an antigen-binding sequence comprising (7a) CDR1 comprising SEQ ID NO:53, (7b) CDR2 comprising SEQ ID NO:54, and (7c) CDR3 comprising SEQ ID NO:55;
(8) an antigen-binding sequence comprising (8a) CDR1 comprising SEQ ID NO:56, (8b) CDR2 comprising SEQ ID NO:57, and (8c) CDR3 comprising SEQ ID NO:58;
(9) an antigen-binding sequence comprising (9a) CDR1 comprising SEQ ID NO:59, (9b) CDR2 comprising SEQ ID NO:60, and (9c) CDR3 comprising SEQ ID NO:61; and
(10) an antigen-binding sequence comprising (10a) CDR1 comprising SEQ ID NO:62, (10b) CDR2 comprising SEQ ID NO:63, and (10c) CDR3 comprising SEQ ID NO: 64.

In some embodiments of the ADC, each of the long chains comprises the sequence of SEQ ID NO:25 and each of the short chains comprises the sequence of SEQ ID NO:26. In some embodiments, each of the long chains comprises the sequence of SEQ ID NO:26 and each of the short chains comprises the sequence of SEQ ID NO:25. In some embodiments, each of the long chains comprises the sequence of SEQ ID NO:27 and each of the short chains comprises the sequence of SEQ ID NO:28. In some embodiments, each of the long chains comprises the sequence of SEQ ID NO:28 and each of the short chains comprises the sequence of SEQ ID NO: 27. In some embodiments, each of the long chains comprises the sequence of SEQ ID NO:29 and each of the short chains comprises the sequence of SEQ ID NO:30. In some embodiments, each of the long chains comprises the sequence of SEQ ID NO:30 and each of the short chains comprises the sequence of SEQ ID NO:29. In some embodiments, each of the long chains comprises the sequence of SEQ ID NO:31 and each of the short chains comprises the sequence of SEQ ID NO:32. In some embodiments, each of the long chains comprises the sequence of SEQ ID NO:32 and each of the short chains comprises the sequence of SEQ ID NO:31. In some embodiments, each of the long chains comprises the sequence of SEQ ID NO:33 and each of the short chains comprises the sequence of SEQ ID NO:34. In some embodiments, each of the long chains comprises the sequence of SEQ ID NO:34 and each of the short chains comprises the sequence of SEQ ID NO:33.

In some embodiments of the ADC, each of the long chains comprises an antigen-binding sequence comprising three CDR regions respectively comprising the sequences of SEQ ID NO: 35, SEQ ID NO:36 and SEQ ID NO:37, and each of the short chains comprises an antigen-binding sequence comprising three CDR regions respectively comprising the sequences of SEQ ID NO: 38, SEQ ID NO:39 and SEQ ID NO:40. In some embodiments of the ADC, each of the short chains comprises an antigen-binding sequence comprising three CDR regions respectively comprising the sequences of SEQ ID NO:35, SEQ ID NO:36 and SEQ ID NO:37, and each of the long chains comprises an antigen-binding sequence comprising three CDR regions respectively comprising the sequences of SEQ ID NO:38, SEQ ID NO:39 and SEQ ID NO:40.

In some embodiments of the ADC, each of the long chains comprises an antigen-binding sequence comprising three CDR regions respectively comprising the sequences of SEQ ID NO: 41, SEQ ID NO:42 and SEQ ID NO:43, and each of the short chains comprises an antigen-binding sequence comprising three CDR regions respectively comprising the sequences of SEQ ID NO: 44, SEQ ID NO:45 and SEQ ID NO:46. In some embodiments of the ADC, each of the short chains comprises an antigen-binding sequence comprising three CDR regions respectively comprising the sequences of SEQ ID NO:41, SEQ ID NO:42 and SEQ ID NO:43, and each of the long chains comprises an antigen-binding sequence comprising three CDR regions respectively comprising the sequences of SEQ ID NO:44, SEQ ID NO:45 and SEQ ID NO:46.

In some embodiments of the ADC, each of the long chains comprises an antigen-binding sequence comprising three CDR regions respectively comprising the sequences of SEQ ID NO: 47, SEQ ID NO:48 and SEQ ID NO:49, and each of the short chains comprises an antigen-binding sequence comprising three CDR regions respectively comprising the sequences of SEQ ID NO: 50, SEQ ID NO:51 and SEQ ID NO:52. In some embodiments of the ADC, each of the short chains comprises an antigen-binding sequence comprising three CDR regions respectively comprising the sequences of SEQ ID NO:47, SEQ ID NO:48 and SEQ ID NO:49, and each of the long chains comprises an antigen-binding sequence comprising three CDR regions respectively comprising the sequences of SEQ ID NO:50, SEQ ID NO:51 and SEQ ID NO:52.

In some embodiments of the ADC, each of the long chains comprises an antigen-binding sequence comprising three CDR regions respectively comprising the sequences of SEQ ID NO: 53, SEQ ID NO:54 and SEQ ID NO:55, and each of the short chains comprises an antigen-binding sequence comprising three CDR regions respectively comprising the sequences of SEQ ID NO: 56, SEQ ID NO:57 and SEQ ID NO:58. In some embodiments of the ADC, each of the short chains comprises an antigen-binding sequence comprising three CDR regions respectively comprising the sequences of SEQ ID NO:53, SEQ ID NO:54 and SEQ ID NO:55, and each of the long chains comprises an antigen-binding sequence comprising three CDR regions respectively comprising the sequences of SEQ ID NO:56, SEQ ID NO:57 and SEQ ID NO:58.

In some embodiments of the ADC, each of the long chains comprises an antigen-binding sequence comprising three CDR regions respectively comprising the sequences of SEQ ID NO: 59, SEQ ID NO:60 and SEQ ID NO:61, and each of the short chains comprises an antigen-binding sequence comprising three CDR regions respectively comprising the sequences of SEQ ID NO: 62, SEQ ID NO:63 and SEQ ID NO:64. In some embodiments of the ADC, each of the short chains comprises an antigen-binding sequence comprising three CDR regions respectively comprising the sequences of SEQ ID NO:59, SEQ ID NO:60 and SEQ ID NO:61, and each of the long chains comprises an antigen-binding sequence comprising three CDR regions respectively comprising the sequences of SEQ ID NO:62, SEQ ID NO:63 and SEQ ID NO:64.

In some embodiments of the ADC, each of the short chains comprises a sequence selected from the group consisting of SEQ ID NO:2, SEQ ID NO:4, SEQ ID NO:6, SEQ ID NO: 8, SEQ ID NO:10, SEQ ID NO:12, SEQ ID NO:14, SEQ ID NO:16, SEQ ID NO:18, SEQ ID NO: 20, SEQ ID NO:22, and SEQ ID NO:24.

In some embodiments of the ADC, each of the long chains comprises a sequence selected from the group consisting of SEQ ID NO:1, SEQ ID NO:3, SEQ ID NO:5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO:21, and SEQ ID NO:23.

In some embodiments of the ADC, the long chains each comprise a sequence of SEQ ID NO: 1 and the short chains each comprise a sequence of SEQ ID NO:2. In some embodiments of the ADC, the long chains each comprise a of SEQ ID NO:3 and the short chains each comprise a sequence of SEQ ID NO:4. In some embodiments of the ADC, the long chains each comprise a sequence of SEQ ID NO:5 and the short chains each comprise a sequence of SEQ ID NO:6. In some embodiments of the ADC, the long chains each comprise a sequence of SEQ ID NO:7 and the short chains each comprise a sequence of SEQ ID NO:8. In some embodiments of the ADC, the long chains each comprise a sequence of SEQ ID NO:9 and the short chains each comprise a sequence of SEQ ID NO:10.

In some embodiments of the ADC, the long chains each comprise a sequence of SEQ ID NO: 11 and the short chains each comprise a sequence of SEQ ID NO: 12. In some embodiments of the ADC, the long chains each comprise a of SEQ ID NO: 13 and the short chains each comprise a sequence of SEQ ID NO:14. In some embodiments of the ADC, the long chains each comprise a sequence of SEQ ID NO: 15 and the short chains each comprise a sequence of SEQ ID NO: 16. In some embodiments of the ADC, the long chains each comprise a sequence of SEQ ID NO: 17 and the short chains each comprise a sequence of SEQ ID NO: 18. In some embodiments of the ADC, the long chains each comprise a sequence of SEQ ID NO: 19 and the short chains each comprise a sequence of SEQ ID NO:20. In some embodiments of the ADC, the long chains each comprise a sequence of SEQ ID NO:21 and the short chains each comprise a sequence of SEQ ID NO:22. In some embodiments of the ADC, the long chains each comprise a sequence of SEQ ID NO:23 and the short chains each comprise a sequence of SEQ ID NO:24.

In some embodiments, the present disclosure provides an ADC which comprises a monoclonal antibody conjugated to a cytotoxic drug by a chemical linker, wherein the monoclonal antibody comprises two identical long chains and four identical short chains, wherein:
(a) each of the long chains comprises the sequence of SEQ ID NO: 1 and each of the short chains comprises the sequence of SEQ ID NO:2; or
(b) each of the long chains comprises the sequence of SEQ ID NO:3 and each of the short chains comprises the sequence of SEQ ID NO:4; or
(c) each of the long chains comprises the sequence of SEQ ID NO:5 and each of the short chains comprises the sequence of SEQ ID NO:6; or
(d) each of the long chains comprises the sequence of SEQ ID NO:7 and each of the short chains comprises the sequence of SEQ ID NO:8; or
(e) each of the long chains comprises the sequence of SEQ ID NO:9 and each of the short chains comprises the sequence of SEQ ID NO: 10; or
(f) each of the long chains comprises the sequence of SEQ ID NO: 11 and each of the short chains comprises the sequence of SEQ ID NO: 12; or
(g) each of the long chains comprises the sequence of SEQ ID NO: 13 and each of the short chains comprises the sequence of SEQ ID NO: 14; or
(h) each of the long chains comprises the sequence of SEQ ID NO: 15 and each of the short chains comprises the sequence of SEQ ID NO: 16; or
(i) each of the long chains comprises the sequence of SEQ ID NO: 17 and each of the short chains comprises the sequence of SEQ ID NO:18; or
(j) each of the long chains comprises the sequence of SEQ ID NO: 19 and each of the short chains comprises the sequence of SEQ ID NO:20; or
(k) each of the long chains comprises the sequence of SEQ ID NO:21 and each of the short chains comprises the sequence of SEQ ID NO:22; or
(l) each of the long chains comprises the sequence of SEQ ID NO:23 and each of the short chains comprises the sequence of SEQ ID NO:24.

In some embodiments of the ADC, the second segment is connected to the remainder of the long chain via a flexible peptide linker comprising an amino sequence of (GₓS)_{y}, where x is a whole number between 1 and 6 inclusive and y is a whole number between 1 and 8 inclusive.

In some embodiments, the antigen is HER2, EGFR, cMet or other antigens, and the ADC contains four antigen-binding fragment domains all specifically bind to these antigens.

In some embodiments of the ADC, the cytotoxic drug is selected from the group consisting of monomethyl auristatin E (MMAE), monomethyl auristatin F (MMAF), auristatin E, auristatin F, maytansine DM1 and DM4, maytansinol, sandramycin, pyrrolobenzodiazepine dimer, anthracyclines, calicheamicin, dolastatin 10, duocarmycin, doxorubicin, thailanstatin A, uncialamycin, amanitins, ricin, diphtheria toxin, eribulin, ¹³¹I, interleukins, tumor necrosis factors, chemokines, and nanoparticles. In one embodiment, the cytotoxic drug is MMAE. In one embodiment, the cytotoxic drug is MMAF. In one embodiment, the cytotoxic drug is DM1. In one embodiment, the cytotoxic drug is DM4.

The chemical linker linking the antibody portion and the cytotoxic drug can be cleavable or non-cleavable. In some embodiments, the linker comprises a PEGn where n is between 1 and 20 (i.e., having 1 to 20 repeat units (CH₂CH₂O)). In some embodiments, the chemical linker further comprises a peptide linker connected to the PEGn spacer. In some embodiments, the chemical linker comprises a peptide linker but does not comprise the PEGn spacer. The peptide linker can be selected from the group consisting of 6-maleimidocaproyl (MC), maleimidopropionyl (MP), valine-citrulline (val-cit), alanine-phenylalanine (ala-phe), p-aminobenzyloxycarbonyl (PAB), N-succinimidyl 4-(2-pyridylthio) valerate (SPP), N-succinimidyl 4-(N-maleimidomethyl)-cyclohexane-1-carboxylate (SMCC), N-succinimidyl (4-iodo-acetyl) aminobenzoate (SIAB), 6-maleimidocaproyl-val-cit-p-aminobenzyloxycarbonyl (MC-vc-PAB), val-cit-PABC, N-succinimidyl-4-(2-pyridyldithio) butanoate (SPDB), N-succinimidyl 3-(pyridin-2-yldithio)-propionate (SPDP), Phe-Lys (Fmoc)-PAB, Aloc-D-Ala-Phe-Lys (Aloc)-PAB-PNP, Boc-Phe-(Alloc) Lys-PAB-PNP, and perfluorophenyl 3-(pyridine-2-yldisulfanyl) propanoate.

In some embodiments of the ADC, the drug-linker can be attached to tetrabody through cysteine coupling, amine coupling, terminal coupling, enzymatic coupling, or carbohydrate coupling.

In some embodiments of the ADC, the drug-to-antibody ratio is between 1 and 12 inclusive. In some embodiments, the drug-to-antibody ratio is about 3 or 4.

The present disclosure also provides a pharmaceutically acceptable salt thereof, of the various embodiments of the ADCs as described herein.

In a further aspect, the present disclosure provides a pharmaceutical composition comprising the ADC, or the pharmaceutically acceptable salt thereof, and a pharmaceutically acceptable carrier.

In a further aspect, the present disclosure provides a method of treating a proliferative disease, disorder, or condition, by administering to a subject an effective amount of the pharmaceutical composition described herein. The proliferative disease may be cancers, such as colon cancer, rectal cancer, lung cancer, breast cancer, ovarian cancer, cervical cancer, bladder cancer, esophageal cancer, sarcoma, and head and neck cancer. The present disclosure also provides a method of treating an EGRF, HER2, or c-MET associated disease, disorder or condition, such as a proliferative disease, disorder or condition, e.g., cancer.

In another aspect, the present disclosure provides nucleic acid molecules encoding the antibodies, or antigen-binding portions, of the invention. Recombinant expression vectors which include nucleic acids encoding antibodies of the invention, and host cells transfected with such vectors, are also encompassed by the invention, as are methods of making the antibodies of the invention by culturing such host cells, e.g., an expression vector comprising a nucleotide sequence encoding the variable and constant regions of the heavy and light chains of antibody 2F8 produced by the hybridoma.

The present disclosure also provides mammalian cells capable of producing the monoclonal antibodies of the invention that specifically bind to the intended targets. In one embodiment, the hybridoma includes a B cell obtained from a transgenic non-human animal, e.g., a transgenic mouse having a genome comprising a human heavy chain transgene and a human light chain transgene encoding all or a portion of an antibody of the invention, fused to an immortalized cell.

The term “about” as used herein with reference to a certain given value or quantity herein means a range of up to 25% deviation from (greater or smaller than) the given value.

As used herein, the term “cancer” is intended to encompass both non-metastatic cancer and metastatic cancer. In this context, treating cancer involves treatment of both primary tumors and tumor metastases.

As used herein, “treatment” refers to clinical intervention that attempts to alter the natural course of a treated individual, which can include the prevention of recurrence or relapse of disease, the alleviation of symptoms, the weakening of any direct or indirect pathological consequences of disease, the prevention of metastasis, the reduction of disease progression rate, the improvement or alleviation of disease status, and the elimination or improvement of prognosis.

In the present disclosure, “subject” refers to mammal, such as cattle, cats, dogs, and horses, primates, mice and rats. In certain embodiments, the mammal refers to a human.

In the present disclosure, “effective amount” refers to an amount effective to achieve the desired therapeutic or prophylactic effect at the desired dose and time. The “therapeutically effective amount” of a substance may vary depending on factors such as disease state, age, gender and body weight of an individual and the ability of the substance/molecule to elicit a desired response in the individual. In the case of cancer, the therapeutically effective amount of an ADC can reduce the number of cancer cells; reduce the tumor volume; inhibit the cancer cells infiltrating into the surrounding organs; inhibit tumor metastasis; inhibit the growth of tumor; and/or alleviate to some extent one or more symptoms associated with cancer. For the prophylaxis or treatment of the disease, the appropriate dosage of the ADC of the present disclosure will depend on the type of disease to be treated, the type of the antibody-drug conjugate, the severity and progression of the disease, the administration of the ADC, etc. Suitably, the antibody-drug conjugate is administered to the patient either once or through a series of treatments. Depending on the type and severity of the disease, the dose administered to the patient may be about 1 μg/kg to 100 mg/kg (e.g., 0.1 mg/kg to 20 mg/kg) of the ADC, for example or by one or more separate administrations or by continuous infusion. Depending on the factors described above, the typical daily dose may range from about 1 μg/kg to 100 mg/kg or more. For repeated administrations for several days or more, depending on the conditions, the treatment is usually continued until the desired inhibition of symptoms appears. An exemplary dose of the antibody-drug conjugate may range from about 0.05 mg/kg to about 10 mg/kg. As such, the antibody-drug conjugate of one or more doses of about 0.5 mg/kg. 2.0 mg/kg, 4.0 mg/kg or 10 mg/kg (or any combination thereof) may be administered to the patient. Such doses may be administered intermittently, for example, weekly or every three weeks (e.g., such that the patient receives about 2 to about 20 doses, or, for example, about 6 doses of the antibody-drug conjugate). A higher initial loading dose may be administered, followed by one or more doses of lower dose. The process of this therapy is easily monitored by conventional techniques and assays.

“Pharmaceutically acceptable carriers” include, when used in the present disclosure, pharmaceutically acceptable carriers, excipients or stabilizers, which are non-toxic to the cells or mammals to which they are exposed at the dosage and concentration employed. Typically, a physiologically acceptable carrier is a pH buffered aqueous solution. Examples of physiologically acceptable carriers include buffers such as phosphates, citrates and other organic acids: antioxidants including ascorbic acid; low molecular weight (less than about 10 residues) polypeptides; proteins such as serum albumin, gelatin or immunoglobulins; hydrophilic polymers such as polyvinylpyrrolidone; amino acids such as glycine, glutamine, asparagine, arginine or lysine; monosaccharides, disaccharides and other carbohydrates, including glucose, mannose, sucrose, trehalose or dextrin; chelating agents such as EDTA; sugar alcohols such as mannitol or sorbitol; salt-forming counterions such as sodium; and/or nonionic surfactants.

In the present disclosure, the drug load or drug to antibody ratio, i.e., the average number of cytotoxic drug modules of each antibody in the ADC can range from 1 to 12 drug per antibody, for example, about 1, about 2, about 3, about 4, about 5, about 6, about 7, about 8, about 9, about 10, about 11, or about 12. Different attachment sites can be used, e.g., through interchain disulfide bonds between heavy and light chains, through interchain disulfide bonds between heavy chains in hinge region, on engineered cysteine, on selected Lys residues on each chain, on carbohydrate, on terminal residues. The average number of drug modules per antibody from the ADC preparation of coupling reaction can be verified by conventional means, such as mass spectrometry, ELISA assay, and HPLC. In certain embodiments, the ADCs of the present disclosure can have a drug load ranging from 1 to about 8; from about 4 to about 12, from about 2 to about 6; from about 3 to about 5; from about 4 to about 5; from about 3.5 to about 4.5; about 4. In certain embodiments, to produce reactive thiol group for the linking of the linker to the antibody, the antibody may be reduced under partial or complete reductive conditions to produce a reactive cysteine thiol group.

In the present disclosure, the pharmaceutically acceptable salts of the ADCs include acid addition salts of inorganic acids, carboxylic acids and sulfonic acids, for example, salts of the following acids: hydrochloric acid, hydrobromic acid, sulfuric acid, phosphoric acid, methanesulfonic acid, ethanesulfonic acid, benzenesulfonic acid, toluenesulfonic acid, naphthalene disulfonic acid, acetic acid, trifluoroacetic acid, propionic acid, lactic acid, tartaric acid, malic acid, citric acid, fumaric acid, maleic acid and benzoic acid.

The pharmaceutically acceptable salts of the antibody-drug conjugates of the present disclosure also include salts of conventional bases, for example alkali metal salts (e.g., sodium salts and potassium salts), alkaline earth metal salts (e.g., calcium salts and magnesium salts) and ammonium salts derived from ammonia or organic amines containing from 1 to 16 carbon atoms, in which the organic amines are, for example, ethylamine, diethylamine, triethylamine, ethyl diisopropylamine, monoethanolamine, diethanolamine, triethanolamine, dicyclohexylamine, dimethylaminoethanol, procaine, dibenzamide, N-methylpiperidine, N-methylmorpholine, arginine, lysine and 1,2-ethylenediamine.

The target protein-associated tumors that can be treated with the ADCs of the invention include tumors with target overexpression. For example, EGFR associated tumors include respiratory tract tumors (e.g., small cell carcinoma and non-small cell carcinoma, bronchial carcinoma); tumors of digestive tracts (e.g., esophagus, stomach, gallbladder, small intestine, large intestine, rectum): tumors of endocrine and exocrine glands (e.g., thyroid and parathyroid, pancreas and salivary glands); tumors of head and neck regions (e.g., larynx, hypopharynx, nasopharynx, oropharynx, lips, mouth, tongue and esophagus); and/or gliomas. HER2-associated tumors include bladder cancers, esophageal cancers, breast cancers, cervical cancers, gastric adenocarcinomas, etc.

The ADCs of the present disclosure may be used in combination with a known chemotherapeutic agent for the treatment of tumors. The chemotherapeutic agent can be, for example, Adriamycin, cyclophosphamide and taxane [Taxol and Taxotere], Xeloda, Gemzar, Navelbine, Tamoxifen, aromatase inhibitors (Arimidex, Femara, Aromasin). 5-FU plus folinic acid, camptosar, oxaliplatin, cisplatin, carboplatin, estramustine, Novantrone, prednisone, Oncovin, etc., or a combination thereof.

### Examples

A summary list of the tetravalent antibodies prepared is shown in Table 1 below.

TABLE 1

| | | SEQ | SEQ |
|---|---|---|---|
| | | ID | ID |
| | | No. | No. |
| | Protein Structure | (long | (short |
| ID | (Long/Short Chain) | chain) | chain) |
| BB01H | VHCH1Fc(g1)(G₃S)₄VHCH1/ | 1 | 2 |
| | VLCL(κ) | | |
| BB01E | VHCH1Fc(g2)(G₃S)₄VHCH1/ | 3 | 4 |

VLCL(λ)

| | | | |
|---|---|---|---|
| BB02E | VHCH1Fc(g1)(G₃S)₄VHCH1/ | 5 | 6 |
| | VLCL(κ) | | |
| BB03E | VHCH1Fc(g1)(G₃S)₄VHCH1/ | 7 | 8 |
| | VLCL(κ) | | |
| BB04E | VHCH1Fc(g2)(G₃S)₁VHCH1/ | 9 | 10 |
| | VLCL(κ) | | |
| BB05E | VLCL(κ)Fc(g1)(G₃S)₄ | 11 | 12 |
| | VLCL(κ)/VHCH1 | | |
| BB02H | VHCH1Fc(g1)(G₃S)₄VHCH1/ | 13 | 14 |
| | VLCL(κ) | | |
| BB03H | VLCL(κ)Fc(g1)(G₃S)₄ | 15 | 16 |
| | VLCL(κ)/VHCH1 | | |
| BB04H | VLCL(κ)Fc(g1)(G₃S)₄ | 17 | 18 |
| | VLCL(κ)/VHCH1 | | |
| BB06E | VLCL(κ)Fc(g1)(G₃S)₄ | 19 | 20 |
| | VLCL(κ)/VHCH1 | | |
| BB01M | VHCH1Fc(g1)(G₃S)₄VHCH1/ | 21 | 22 |
| | VLCL(κ) | | |
| BB02M | VLCL(κ)Fc(g1)(G₃S)₄ | 23 | 24 |
| | VLCL(κ)/VHCH1 | | |

### 1. Preparation of Tetravalent Antibodies

Example tetravalent antibodies were designed and synthesized using sequences in Table 1 fused with constant isotype of choice. In some examples, certain point mutations are introduced for specific purpose. Two types of tetravalent antibodies were prepared, conventional and twisted formats. Each tetravalent antibody contains two long chains and four short chains. For conventional format, the sequences of the long chain contain a full-length heavy chain and an intact VH-CH₁ fragment of the same antibody fused to the C-terminus of Fc through a flex (GS) linker. The short chain sequences are the same as the light chain of the same antibody.

For twisted format, the sequences of the long chains contain a full length light chain (VL-CL) directly fused to the N-terminus of Fc (CH2-CH3) and another full length light chain (VL-CL) (of the same antibody) fused to the C-terminus of Fc through a flex (GS) linker. The sequences of the short chains contain the full length of VH-CH1 of the same antibody.

For expression of the engineered tetravalent antibodies, codon optimization and gene synthesis were performed. Specific full-length long chain and short chain DNA were each cloned into a separate pcDNA3 plasmid. HEK293 cell transient transfection of the paired plasmids and one-step Protein A purification was used to prepare sufficient amount of proteins for testing. Example tetravalent antibodies:
(a) BB01H: BB01H is made in conventional format of IgG1K isotype, where its long chain contains: VH-CH1-CH2-CH3-(G₃S)₄-VH-CH1, and short chain contains: VL-CL (κ). As used in the sequence of a tetravalent antibody described herein, VH, VL, CH1, CH2, CH3 refer to the domains or fragments of the precursor antibody from which the tetravalent antibody is constructed. Sequence of the long chain of BB01H is shown as SEQ ID NO:1. Sequence of the short chain of BB01H is shown as SEQ ID NO:2.

(b) BB01E: BB01E is prepared in conventional format of IgG2λ isotype, where its long chain contains VH-CH1-CH2-CH3-(G₃S)₄-VH-CH1 (CH2-CH3 of IgG2 is also referred to as Fc (g2)), and short chain contains VL-CL (λ). Sequence of the long chain of BB01E is shown as SEQ ID NO:3. Sequence of short chain of BB01E is shown as SEQ ID NO: 4.

(c) BB04E: BB04E is made in conventional format of IgG2κ isotype, where its long chain contains: VH-CH1-CH2-CH3-(G₃S)₁-VH-CH1, and short chain contains: VL-CL (κ). Sequence of the long chain of BB04E is shown as SEQ ID NO:9. Sequence of the short chain of BB04E is shown as SEQ ID NO:10.

(d) BB05E: BB05E is made in twisted format of IgG₁κ isotype, where its long chain contains: VL-CL (κ)-CH2-CH3 (G₃S)₄-VL-CL (κ), and short chain contains: VH-CH1 (IgG1). Sequence of the long chain of BB05E is shown as SEQ ID NO:11. Sequence of the short chain of BB05E is shown as SEQ ID NO: 12.

### 2. Preparation of Tetravalent ADCs

Tetravalent antibodies (BB01E, BB02E, BB03E, BB04E, BB05E) and control bivalent antibody (Ctl) are conjugated to the cytotoxic agents (e.g., MMAE, DM1) via a linker. Antibody in phosphate buffer at neutral pH is added TCEP for partial reduction. Drug-linker (MC-val-cit-PAB-MMAE or MC-DM1) in DMA is added and allowed to react with tetrabody to obtain desired drug-to-antibody ratio (DAR).

Tetravalent antibody in phosphate buffer is added OSu-Glu-val-cit-PAB-MMAE in DMA, and allowed to react to obtain desired DAR.

### 3. Characterization of Tetravalent Antibodies and Tetravalent ADCs

### (a) Hydrophobic Interaction Chromatography (HIC): HIC is Used for the Evaluation of Drug Distribution and Molar Ratio of Drug and Antibody in ADC.

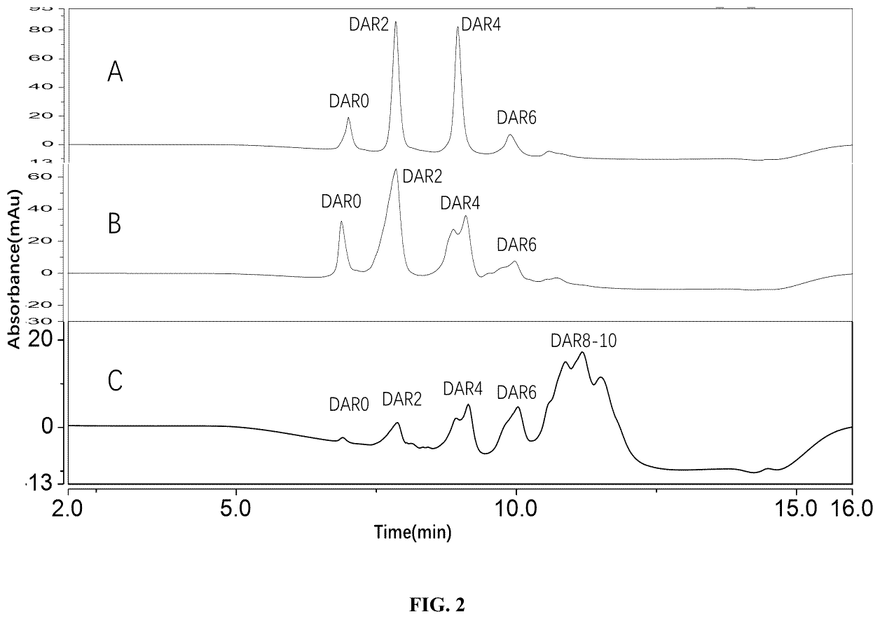

Ctl-MMAE (bivalent ADC), BB01E-MMAE (tetravalent ADC) with low DAR, and BB01E-MMAE with high DAR were analyzed by HIC, and the representative HIC chromatograms are presented in FIG. 2. For Ctl-MMAE (bivalent ADC) and BB01E-MMAE with low DAR, the main peaks are antibodies linked to 2 and 4 drug molecules (DAR2, DAR4, respectively), and the minor peaks are antibodies (DAR0) and antibodies linked to 6 and 8 drug molecules (DAR6, DAR8). The results indicate BB01E-MMAE and Ctl-MMAE have similar drug distribution and DAR values. BB01E-MMAE with high DAR mainly contains antibodies linked to 8 and 10 drug molecules (DAR8, DAR10).

### (b) Binding

### (b1) Binding to Recombinant Expressed Target Proteins.

ELISA assay is used to exam and compare the target binding capabilities (to EGFR or HER2) between the tetravalent antibodies and control bivalent antibodies. Tetravalent antibody samples or control antibodies diluted in 5-fold serial dilutions starting from 20 μg/mL and 8 dilutions total, were coated onto 96-well plates and the plates were incubated at 4° C. overnight. Using HRP-labeled human target protein (EGFR or HER2 at 5 ng/mL) as a detection agent and TMB for colorimetric reaction, the plates read at 450/650 nm for absorbance on Microplate Reader (Molecular Devices, SpectraMax 190) and data analysis was performed using a dose response curve format four parameters logistic model.

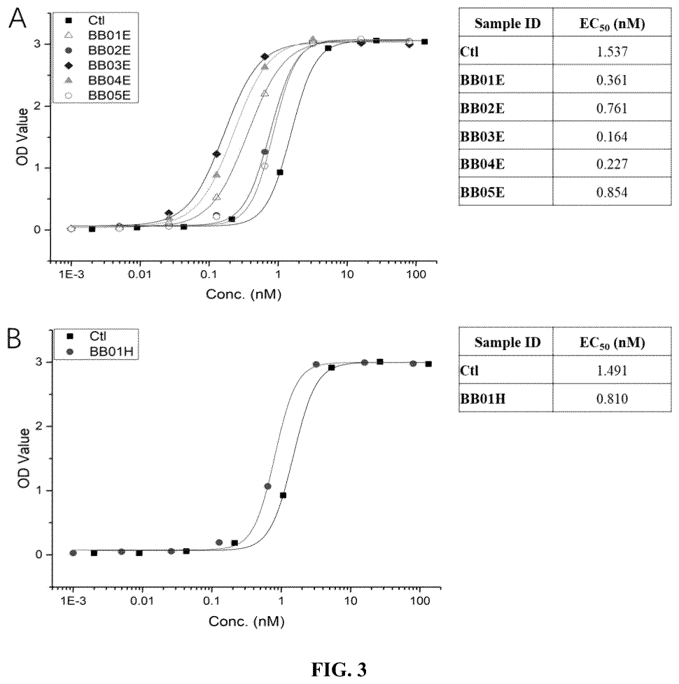

The results in FIG. 3 showed that all tetravalent antibody variants bound to their specific target proteins with enhanced capabilities in comparison with the control bivalent antibodies.

### (b2) Binding to EGFR-Expressing Cancer Cells

FACS assay on several cancer cell lines (MDA-MB-468, NUGC3) with different levels of surface EGFR was used to analyze binding activity of tetravalent antibodies to EGFR-expressing cell lines and compared with that of the bivalent control. Briefly, single cell suspension was first stained with tetravalent antibody samples or control antibody at saturate concentration (10 μg/mL) and then stained with FITC-labeled anti-human kappa antibody as a secondary antibody.

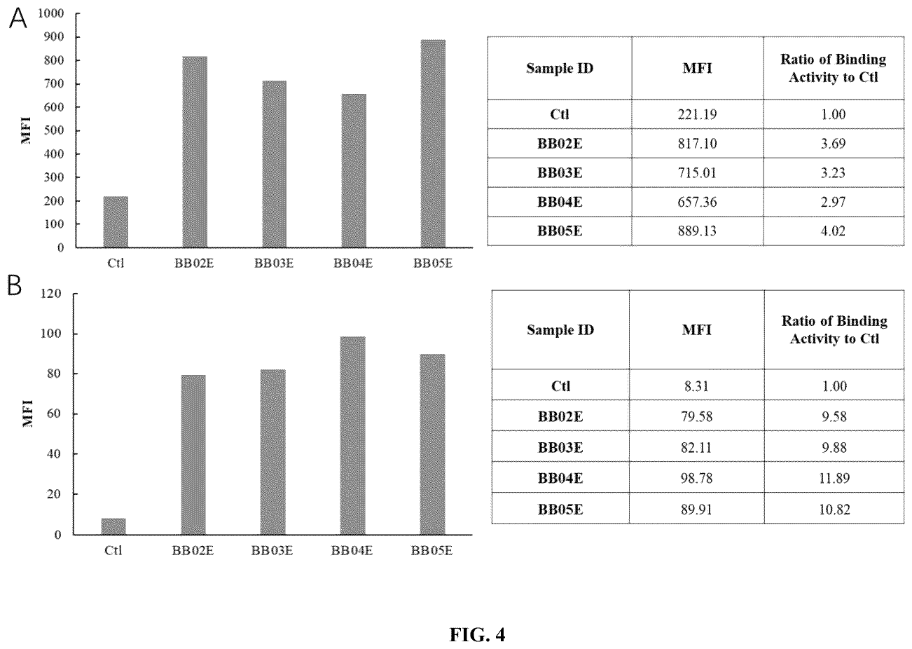

The results in FIG. 4 showed that the mean fluorescent intensity (MFI) of tetravalent anti-EGFR antibodies bound onto EGFR high-expressing MDA-MB468 cells were 3-4-fold higher than that of bivalent control. The MFI differences were even greater on EGFR low-expressing NUGC3 cells (nearly 10-fold differences). Although the two more copies of kappa on tetravalent antibody might contribute to a higher signal (two-fold), the significantly higher MFI (3-10 fold) of tetravalent antibody staining suggest that the four targeting arms on tetravalent format offered significantly more efficient synergy when binding to targets than the two arms on bivalent format. This synergistic effect was more pronounce when binding to EGFR low-expressing cells than to EGFR high-expressing cells.

### (c) Internalization

Tetravalent antibody induced internalization of antibody-target complex was analyzed on several cancer cell lines (MDA-MB-468, NUGC3) with different levels of surface EGFR and compare with that of the bivalent control. To do the assay, single cell suspensions were first incubated with antibody samples (10 μg/mL) on ice for 1 hr and then washed to remove unbound antibodies. While one aliquot remained on ice, the rest were incubated at 37° C. for different period of time (0.5, 1, 2 and 4 hrs). At the end of each defined incubation time, an aliquot of cells was taken out of the 37° C. incubator and put on ice. When the longest incubation finished, all aliquots were stained with FITC-labeled secondary antibody and stained samples were analyzed by flow cytometry. Receptor-antibody complex internalization was calculated as percent MFI loss at 37° C. in relative to that on ice after subtracting the background value of MFI derived from the stained control. Percentage internalization was calculated according to: percent of internalization (%)=(MFI_{0 h}−MFI_{other times})/MFI_{0 h}

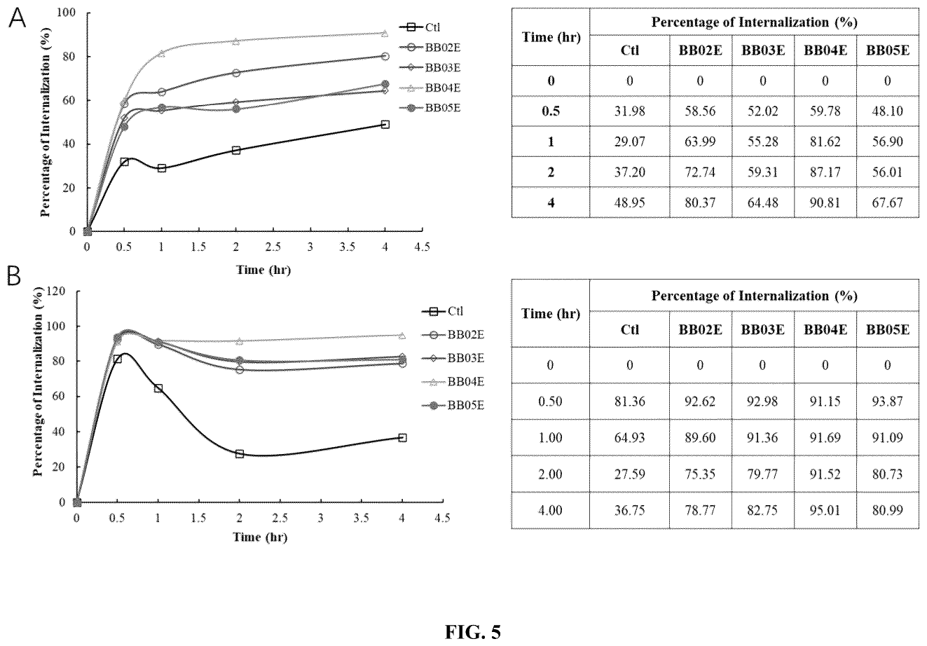

The results in FIG. 5 showed that, on both EGFR high-expressing MDA-MB-468 cells and EGFR low-expressing NUGC3 cells, the percent of internalized tetravalent antibodies was higher than that of the bivalent control, suggesting that the internalization potential of tetravalent antibodies in EGFR-positive human cancer cells was significantly enhanced over that of the bivalent control.

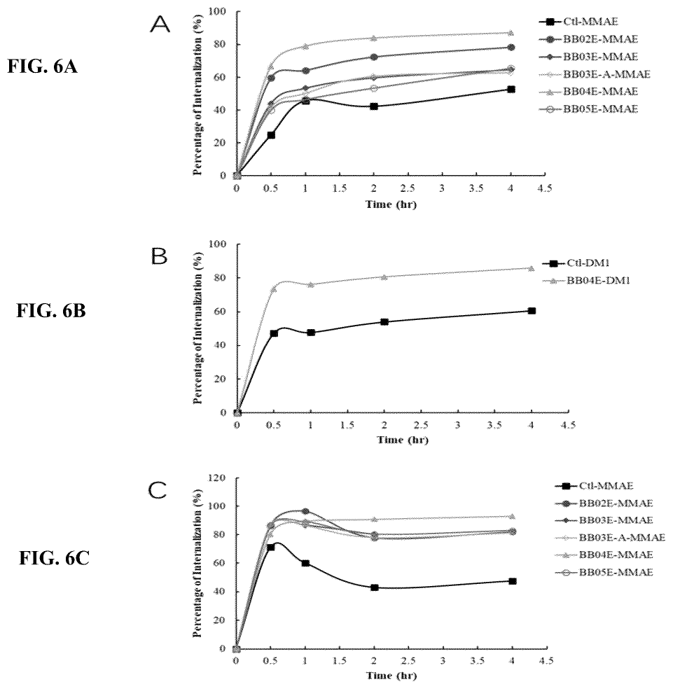

Similar internalization analysis was performed for tetravalent ADC and conventional ADC as controls. In all cases, tetravalent ADC with either MMAE payload or DM1 payload, internalized more efficiently than conventional bivalent ADC (FIGS. 6A-6C).

### (d) Cytotoxicity

To investigate whether the enhanced internalization of tetravalent antibodies and ADCs would translate into more potent cytotoxicity, in vitro cytotoxicity to target expressing cancer cells with varying EGFR expression levels was evaluated in comparison with conventional bivalent-ADC in a colorimetric-based cytotoxic assay. To perform the assay, target cells were seeded into a 96-well flat-bottom tissue culture plate at an optimized cell density for each cell line and incubated at 37° C., 5% CO₂ overnight (16-20 hrs). Serial dilutions of ADC samples were transferred to cell plate and the assay plates were incubated for a defined period of time (3-5 days depend on cell lines) for optimal killing.

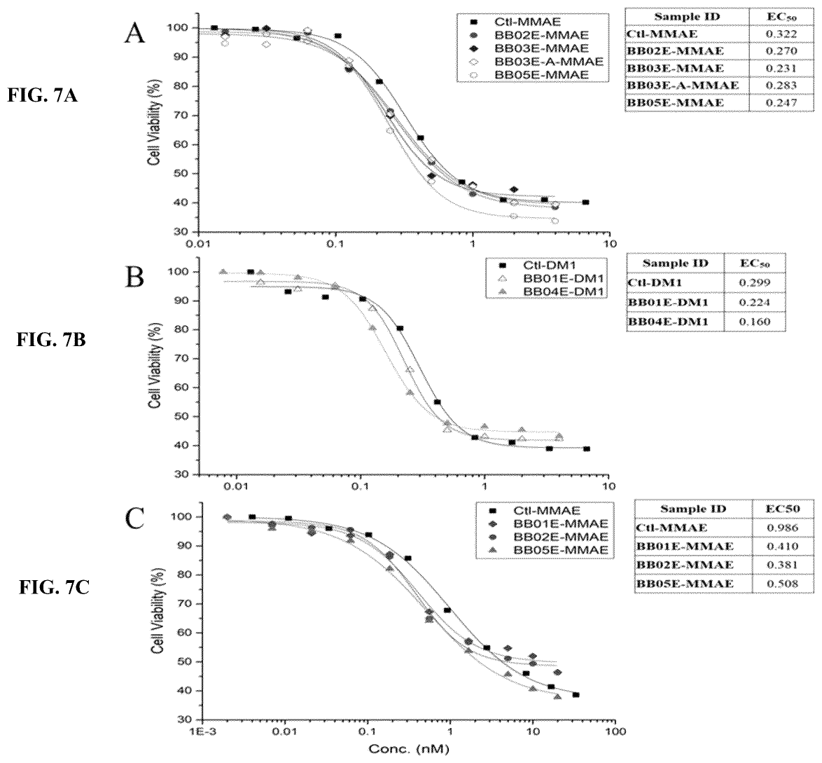

The results in FIGS. 7A-7C showed that various tetravalent ADC product with either MMAE payload or DM1 payload, had more potent cytotoxicity than conventional bivalent ADC to target expressing cells.

While specific embodiments of the present invention have been described in detail, those skilled in the art will understand that various modifications and substitutions can be made to those details according to all teachings that have been disclosed, and all of these changes fall within the scope of the present invention. The full scope of the invention is given by the appended claims and any equivalents thereof.

Sequence Listing
BB01H
Chain 1 (long chain, SEQ ID NO: 1):
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWGQGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPA
PEAAGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGSGGGSGGGSGGGS
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWGQGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSC
Chain 2 (short chain, SEQ ID NO: 2):
DIQMTQSPSSLSASVGDRVTITCRASQDVNTAVAWYQQKPGKAPKLLIYSASFLYSGVP
SRFSGSRSGTDFTLTISSLQPEDFATYYCQQHYTTPPTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
BB01E
Chain 1 (long chain, SEQ ID NO: 3):
QVQLKQSGPGLVQPSQSLSITCTVSGFSLTNYGVHWVRQSPGKGLEWLGVIWSGGNTD
YNTPFTSRLSINKDNSKSQVFFKMNSLQSNDTAIYYCARALTYYDYEFAYWGQGTLVT
VSAASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCCVECPPCPAPPVAG
PSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSR
EEMTKNQVSLTCLVKGFYPSDISVEWESNGQPENNYKTTPPMLDSDGSFFLYSKLTVDK
SRWQQGNVFSCSVMHEALHNHYTQKSLSLSPGKGGGSGGGSGGGSGGGSQVQLKQSG
PGLVQPSQSLSITCTVSGFSLTNYGVHWVRQSPGKGLEWLGVIWSGGNTDYNTPFTSRL
SINKDNSKSQVFFKMNSLQSNDTAIYYCARALTYYDYEFAYWGQGTLVTVSAASTKGP
SVFPLAPCSRSTSESTAALGCLVKDYFPEPVTVSWNSGALTSGVHTFPAVLQSSGLYSLS
SVVTVPSSNFGTQTYTCNVDHKPSNTKVDKTV
Chain 2 (short chain, SEQ ID NO: 4): VL-CL(λ):
DILLTQSPVILSVSPGERVSFSCRASQSIGTNIHWYQQRTNGSPRLLIKYASESISGIPSRFS
GSGSGTDFTLSINSVESEDIADYYCQQNNNWPTTFGAGTKLELKGQPKANPTVTLFPPSS
EELQANKATLVCLISDFYPGAVTVAWKADGSPVKAGVETTKPSKQSNNKYAASSYLSL
TPEQWKSHRSYSCQVTHEGSTVEKTVAPTECS
BB02E
Chain 1 (long chain, SEQ ID NO: 5):
QVQLKQSGPGLVQPSQSLSITCTVSGFSLTNYGVHWVRQSPGKGLEWLGVIWSGGNTD
YNTPFTSRLSINKDNSKSQVFFKMNSLQSDDTAIYYCARALTYYDYEFAYWGQGTLVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKTHTCPPCPAPE
AAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGSGGGSGGGSGGGSQVQ
LKQSGPGLVQPSQSLSITCTVSGFSLTNYGVHWVRQSPGKGLEWLGVIWSGGNTDYNTP
FTSRLSINKDNSKSQVFFKMNSLQSDDTAIYYCARALTYYDYEFAYWGQGTLVTVSAAS
TKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSC
Chain 2 (short chain, SEQ ID NO: 6):
DILLTQSPVILSVSPGERVSFSCRASQSIGTNIHWYQQRTNGSPRLLIKYASESISGIPSRFS
GSGSGTDFTLSINSVESEDIADYYCQQNNNWPTTFGAGTKLELKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
BB03E
Chain 1 (long chain, SEQ ID NO: 7):
QVQLQESGPGLVKPSETLSLTCTVSGGSVSSGDYYWTWIRQSPGKGLEWIGHIYYSGNT
NYNPSLKSRLTISIDTSKTQFSLKLSSVTAADTAIYYCVRDRVTGAFDIWGQGTMVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPEAAGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGKGGGSGGGSGGGSGGGSQVQLQESG
PGLVKPSETLSLTCTVSGGSVSSGDYYWTWIRQSPGKGLEWIGHIYYSGNTNYNPSLKS
RLTISIDTSKTQFSLKLSSVTAADTAIYYCVRDRVTGAFDIWGQGTMVTVSSASTKGPSV
FPLAPSSKSTSGGTAALGCLVKDYFPEPVTVSWNSGALTSGVHTFPAVLQSSGLYSLSSV
VTVPSSSLGTQTYICNVNHKPSNTKVDKRVEPKSC
Chain 2 (short chain, SEQ ID NO: 8):
DIQMTQSPSSLSASVGDRVTITCQASQDISNYLNWYQQKPGKAPKLLIYDASNLETGVPS
RFSGSGSGTDFTFTISSLQPEDIATYFCQHFDHLPLAFGGGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
BB04E
Chain 1 (long chain, SEQ ID NO: 9):
QVQLKQSGPGLVQPSQSLSITCTVSGFSLTNYGVHWVRQSPGKGLEWLGVIWSGGNTD
YNTPFTSRLSINKDNSKSQVFFKMNSLQSDDTAIYYCARALTYYDYEFAYWGQGTLVT
VSAASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCCVECPPCPAPPVAG
PSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSR
EEMTKNQVSLTCLVKGFYPSDISVEWESNGQPENNYKTTPPMLDSDGSFFLYSKLTVDK
SRWQQGNVFSCSVMHEALHNHYTQKSLSLSPGKGGGSQVQLKQSGPGLVQPSQSLSITC
TVSGFSLTNYGVHWVRQSPGKGLEWLGVIWSGGNTDYNTPFTSRLSINKDNSKSQVFFK
MNSLQSDDTAIYYCARALTYYDYEFAYWGQGTLVTVSAASTKGPSVFPLAPCSRSTSES
TAALGCLVKDYFPEPVTVSWNSGALTSGVHTFPAVLQSSGLYSLSSVVTVPSSNFGTQT
YTCNVDHKPSNTKVDKTV
Chain 2 (short chain, SEQ ID NO: 10):
DILLTQSPVILSVSPGERVSFSCRASQSIGTNIHWYQQRTNGSPRLLIKYASESISGIPSRFS
GSGSGTDFTLSINSVESEDIADYYCQQNNNWPTTFGAGTKLELKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
BB05E
Chain 1 (long chain, SEQ ID NO: 11):
DILLTQSPVILSVSPGERVSFSCRASQSIGTNIHWYQQRTNGSPRLLIKYASESISGIPSRFS
GSGSGTDFTLSINSVESEDIADYYCQQNNNWPTTFGAGTKLELKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGECDKTHTCPPCPAPEAAGGPSVFLFPPKP
KDTLMISRTPEVTCVVVDVHEDPEVKFNWYVDGVEVHNAKTKPREEQYNSTYRVVS
VLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNV
FSCSVMHEALHNHYTQKSLSLSPGKGGGSGGGSGGGSGGGSDILLTQSPVILSVSPGERV
SFSCRASQSIGTNIHWYQQRTNGSPRLLIKYASESISGIPSRFSGSGSGTDFTLSINSVESED
IADYYCQQNNNWPTTFGAGTKLELKRTVAAPSVFIFPPSDEQLKSGTASVVCLLNNFYP
REAKVQWKVDNALQSGNSQESVTEQDSKDSTYSLSSTLTLSKADYEKHKVYACEVTHQ
GLSSPVTKSFNRGEC
Chain 2 (short chain, SEQ ID NO: 12):
QVQLKQSGPGLVQPSQSLSITCTVSGFSLTNYGVHWVRQSPGKGLEWLGVIWSGGNTD
YNTPFTSRLSINKDNSKSQVFFKMNSLQSDDTAIYYCARALTYYDYEFAYWGQGTLVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSC
BB02H
Chain 1 (long chain, SEQ ID NO: 13):
EVQLVESGGGLVQPGGSLRLSCAASGFTFTDYTMDWVRQAPGKGLEWVADVNPNSGG
SIYNQRFKGRFTLSVDRSKNTLYLQMNSLRAEDTAVYYCARNLGPSFYFDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKTHTCPPCPAPE
AAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGSGGGSGGGSGGGSEVQ
LVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSC
Chain 2 (short chain, SEQ ID NO: 14):
DIQMTQSPSSLSASVGDRVTITCKASQDVSIGVAWYQQKPGKAPKLLIYSASYRYTGVPS
RFSGSGSGTDFTLTISSLQPEDFATYYCQQYYIYPYTFGQGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
BB03H
Chain 1 (long chain, SEQ ID NO: 15):
DIQMTQSPSSLSASVGDRVTITCKASQDVSIGVAWYQQKPGKAPKLLIYSASYRYTGVPS
RFSGSGSGTDFTLTISSLQPEDFATYYCQQYYIYPYTFGQGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGECDKTHTCPPCPAPEAAGGPSVFLFPPK
PKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNV
FSCSVMHEALHNHYTQKSLSLSPGKGGGSGGGSGGGSGGGSDIQMTQSPSSLSASVGDR
VTITCKASQDVSIGVAWYQQKPGKAPKLLIYSASYRYTGVPSRFSGSGSGTDFTLTISSLQ
PEDFATYYCQQYYIYPYTFGQGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEVT
HQGLSSPVTKSFNRGEC
Chain 2 (short chain, SEQ ID NO: 16)
EVQLVESGGGLVQPGGSLRLSCAASGFTFTDYTMDWVRQAPGKGLEWVADVNPNSGG
SIYNQRFKGRFTLSVDRSKNTLYLQMNSLRAEDTAVYYCARNLGPSFYFDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSC
BB04H
Chain 1 (long chain, SEQ ID NO: 17):
DIQMTQSPSSLSASVGDRVTITCRASQDVNTAVAWYQQKPGKAPKLLIYSASFLYSGVP
SRFSGSRSGTDFTLTISSLQPEDFATYYCQQHYTTPPTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGECDKTHTCPPCPAPEAAGGPSVFLFP
PKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQG
NVFSCSVMHEALHNHYTQKSLSLSPGKGGGSGGGSGGGSGGGSDIQMTQSPSSLSASVG
DRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYAC
EVTHQGLSSPVTKSFNRGEC
Chain 2(short chain, SEQ ID NO: 18):
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWGQGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSC
BB06E
Chain 1 (long chain, SEQ ID NO: 19):
DIQMTQSPSSLSASVGDRVTITCQASQDISNYLNWYQQKPGKAPKLLIYDASNLETGVPS
RFSGSGSGTDFTFTISSLQPEDIATYFCQHFDHLPLAFGGGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGECDKTHTCPPCPAPEAAGGPSVFLFPPK
PKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNV
FSCSVMHEALHNHYTQKSLSLSPGKGGGSGGGSGGGSGGGSDIQMTQSPSSLSASVGDR
VTITCQASQDISNYLNWYQQKPGKAPKLLIYDASNLETGVPSRFSGSGSGTDFTFTISSLQ
PEDIATYFCQHFDHLPLAFGGGTKVEIKRTVAAPSVFIFPPSDEQLKSGTASVVCLLNNFY
PREAKVQWKVDNALQSGNSQESVTEQDSKDSTYSLSSTLTLSKADYEKHKVYACEVTH
QGLSSPVTKSFNRGEC
Chain 2 (short chain, SEQ ID NO: 20):
QVQLQESGPGLVKPSETLSLTCTVSGGSVSSGDYYWTWIRQSPGKGLEWIGHIYYSGNT
NYNPSLKSRLTISIDTSKTQFSLKLSSVTAADTAIYYCVRDRVTGAFDIWGQGTMVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSC
BB01M
Chain 1 (long chain, SEQ ID NO: 21):
EVQLVESGGGLVQPGGSLRLSCAASGYTFTSYWLHWVRQAPGKGLEWVGMlDPSNSD
TRFNPNFKDRFTISADTSKNTAYLQMNSLRAEDTAVYYCATYRSYVTPLDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKTHTCPPCPAPE
AAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGSGGGSGGGSGGGSEVQ
LVESGGGLVQPGGSLRLSCAASGYTFTSYWLHWVRQAPGKGLEWVGMIDPSNSDTRFN
PNFKDRFTISADTSKNTAYLQMNSLRAEDTAVYYCATYRSYVTPLDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSC
Chain 2 (short chain, SEQ ID NO: 22):
DIQMTQSPSSLSASVGDRVTITCKSSQSLLYTSSQKNYLAWYQQKPGKAPKLLIYWAST
RESGVPSRFSGSGSGTDFTLTISSLQPEDFATYYCQQYYAYPWTFGQGTKVEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
BB02M
Chain 1(long chain, SEQ ID NO: 23):
DIQMTQSPSSLSASVGDRVTITCKSSQSLLYTSSQKNYLAWYQQKPGKAPKLLIYWAST
RESGVPSRFSGSGSGTDFTLTISSLQPEDFATYYCQQYYAYPWTFGQGTKVEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGECDKTHTCPPCPAPEAAGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGKGGGSGGGSGGGSGGGSDIQMTQSPS
SLSASVGDRVTITCKSSQSLLYTSSQKNYLAWYQQKPGKAPKLLIYWASTRESGVPSRFS
GSGSGTDFTLTISSLQPEDFATYYCQQYYAYPWTFGQGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
Chain 2(short chain, SEQ ID NO: 24):
EVQLVESGGGLVQPGGSLRLSCAASGYTFTSYWLHWVRQAPGKGLEWVGMIDPSNSD
TRFNPNFKDRFTISADTSKNTAYLQMNSLRAEDTAVYYCATYRSYVTPLDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSC
Antibody Antigen-Binding Site Partial Sequence
Chain 1 (SEQ ID NO: 25) (underlined portions indicate CDR1, CDR2,
CDR3):
EVQLVESGGGLVQPGGSLRLSCAASGFNIKDTYIHWVRQAPGKGLEWVARIYPTNGYTRYADSV
KGRFTISADTSKNTAYLQMNSLRAEDTAVYYCSRWGGDGFYAMDYWGQGTLVTVSS
Chain 2 (SEQ ID NO: 26) (underlined portions indicate CDR1, CDR2,
CDR3):
DIQMTQSPSSLSASVGDRVTITCRASQDVNTAVAWYQQKPGKAPKLLIYSASFLYSGVPSRFSGS
RSGTDFTLTISSLQPEDFATYYCQQHYTTPPTFGQGTKVEIK
Antibody Antigen-Binding Site Partial Sequence
Chain 1 (SEQ ID NO: 27) (underlined portions indicate CDR1, CDR2,
CDR3):
EVQLVESGGGLVQPGGSLRLSCAASGFTFTDYTMDWVRQAPGKGLEWVADVNPNSGGSIYNQR
FKGRFTLSVDRSKNTLYLQMNSLRAEDTAVYYCARNLGPSFYFDYWGQGTLVTVSS
Chain 2 (SEQ ID NO: 28) (underlined portions indicate CDR1, CDR2,
CDR3):
DIQMTQSPSSLSASVGDRVTITCKASQDVSIGVAWYQQKPGKAPKLLIYSASYRYTGVPSRFSGS
GSGTDFTLTISSLQPEDFATYYCQQYYIYPYTFGQGTKVEIK
Antibody Antigen-Binding Site Partial Sequence
Chain 1 (SEQ ID NO: 29) (underlined portions indicate CDR1, CDR2,
CDR3):
QVQLKQSGPGLVQPSQSLSITCTVSGFSLTNYGVHWVRQSPGKGLEWLGVIWSGGNTDYNTPFT
SRLSINKDNSKSQVFFKMNSLQSDDTAIYYCARALTYYDYEFAYWGQGTLVTVSA
Chain 2 (SEQ ID NO: 30) (underlined portions indicate CDR1, CDR2,
CDR3):
DILLTQSPVILSVSPGERVSFSCRASQSIGTNIHWYQQRTNGSPRLLIKYASESISGIPSRFSGS
GSGTDFTLSINSVESEDIADYYCQQNNNWPTTFGAGTKLELK
Antibody Antigen-Binding Site Partial Sequence
Chain 1 (SEQ ID NO: 31) (underlined portions indicate CDR1, CDR2,
CDR3):
QVQLQESGPGLVKPSETLSLTCTVSGGSVSSGDYYWTWIRQSPGKGLEWIGHIYYSGNTNYNPSL
KSRLTISIDTSKTQFSLKLSSVTAADTAIYYCVRDRVTGAFDIWGQGTMVTVSS
Chain 2 (SEQ ID NO: 32) (underlined portions indicate CDR1, CDR2,
CDR3):
DIQMTQSPSSLSASVGDRVTITCQASQDISNYLNWYQQKPGKAPKLLIYDASNLETGVPSRFSGS
GSGTDFTFTISSLQPEDIATYFCQHFDHLPLAFGGGTKVEIK
Antibody Antigen-Binding Site Partial Sequence
Chain 1 (SEQ ID NO: 33) (underlined portions indicate CDR1, CDR2,
CDR3):
EVQLVESGGGLVQPGGSLRLSCAASGYTFTSYWLHWVRQAPGKGLEWVGMIDPSNSDTRFNPN
FKDRFTISADTSKNTAYLQMNSLRAEDTAVYYCATYRSYVTPLDYWGQGTLVTVSS
Chain 2 (SEQ ID NO: 34) (underlined portions indicate CDR1, CDR2,
CDR3):
DIQMTQSPSSLSASVGDRVTITCKSSQSLLYTSSQKNYLAWYQQKPGKAPKLLIYWASTRESGVP
SRFSGSGSGTDFTLTISSLQPEDFATYYCQQYYAYPWTFGQGTKVEIK
CDR Sequences in SEQ ID Nos: 25-34
(SEQ ID NO: 35)
GFNIKDT
(SEQ ID NO: 36)
YPTNGY
(SEQ ID NO: 37)
WGGDGFYAMDY
(SEQ ID NO: 38)
RASQDVNTAVA
((SEQ ID NO: 39)
SASFLYS
(SEQ ID NO: 40)
QQHYTTPPT
(SEQ ID NO: 41)
GFTFTDY
(SEQ ID NO: 42)
NPNSGG
(SEQ ID NO: 43)
NLGPSFYFDY
(SEQ ID NO: 44)
KASQDVSIGVA
(SEQ ID NO: 45)
SASYRYT
(SEQ ID NO: 46)
QQYYIYPYT
(SEQ ID NO: 47)
GFSLTNY
(SEQ ID NO: 48)
WSGGN
(SEQ ID NO: 49)
ALTYYDYEFAY
(SEQ ID NO: 50)
RASQSIGTNIH
(SEQ ID NO: 51)
YASESIS
(SEQ ID NO: 52)
QQNNNWPTT
(SEQ ID NO: 53)
GGSVSSGDY
(SEQ ID NO: 54)
YYSGN
(SEQ ID NO: 55)
DRVTGAFDI
(SEQ ID NO: 56)
QASQDISNYLN
(SEQ ID NO: 57)
DASNLET
(SEQ ID NO: 58)
QHFDHLPLA
(SEQ ID NO: 59)
GYTFTSY
(SEQ ID NO: 60)
DPSNSD
(SEQ ID NO: 61)
YRSYVTPLDY
(SEQ ID NO: 62)
KSSQSLLYTSSQKNYLA
(SEQ ID NO: 63)
WASTRES
(SEQ ID NO: 64)
QQYYAYPWT

1. An antibody-drug conjugate (ADC) or a pharmaceutically acceptable salt thereof, comprising:
a tetravalent monoclonal antibody conjugated to a cytotoxic drug by a chemical linker,
wherein the monoclonal antibody comprises:
two long chains and four short chains, wherein each of the long chains comprises a first segment and a second segment, the first segment located proximal to the N-terminus of the long chain, and the second segment located proximal to the C-terminus of the long chain, each of the first segment and second segment paring with one of the short chains to form an antigen-binding fragment domain, therefore forming four antigen-binding fragment domains having specificity toward a same antigen.

2. The ADC, or the pharmaceutically acceptable salt thereof, of claim 1, wherein the two long chains are identical, and wherein the four short chains are identical.

3. The ADC, or the pharmaceutically acceptable salt thereof, of claim 1, wherein the first segment and the second segment of each of the long chains have identical sequence.

4. The ADC, or the pharmaceutically acceptable salt thereof, of claim 1, wherein the first segment and the second segment of each of the long chains have different sequences.

5. The ADC, or the pharmaceutically acceptable salt thereof, of claim 1, wherein each of the long chains comprises a subsequence having the sequence of CH2-CH3 domain of one of a native human IgG1, IgG2, IgG4 isotypes, the subsequence disposed between the first segment and the second segment.

6. The ADC, or the pharmaceutically acceptable salt thereof, of claim 5, wherein the subsequence has one or more mutations.

7. The ADC, or the pharmaceutically acceptable salt thereof, of claim 1, wherein each of the short chains comprises the sequence of VL-CL (λ) or VL-CL (κ) of a native human IgG isotype.

8. The ADC, or the pharmaceutically acceptable salt thereof, of claim 1, wherein each of the short chains comprises the sequence of VH-CH1, where CH1 is of a human IgG isotype.

9. The ADC, or the pharmaceutically acceptable salt thereof, of claim 1, wherein at least one of the first segment or the second segment of at least one of the long chains comprises a sequence of VL-CL (κ) of a native human IgG1, IgG2, or IgG4 isotype.

10. The ADC, or the pharmaceutically acceptable salt thereof, of claim 1, wherein:
(a) each of the long chains comprises the sequence of SEQ ID NO:1 and each of the short chains comprises the sequence of SEQ ID NO:2; or
(b) each of the long chains comprises the sequence of SEQ ID NO:3 and each of the short chains comprises the sequence of SEQ ID NO:4; or
(c) each of the long chains comprises the sequence of SEQ ID NO:5 and each of the short chains comprises the sequence of SEQ ID NO:6; or
(d) each of the long chains comprises the sequence of SEQ ID NO:7 and each of the short chains comprises the sequence of SEQ ID NO:8; or
(e) each of the long chains comprises the sequence of SEQ ID NO:9 and each of the short chains comprises the sequence of SEQ ID NO:10; or
(f) each of the long chains comprises the sequence of SEQ ID NO:11 and each of the short chains comprises the sequence of SEQ ID NO: 12; or
(g) each of the long chains comprises the sequence of SEQ ID NO: 13 and each of the short chains comprises the sequence of SEQ ID NO:14; or
(h) each of the long chains comprises the sequence of SEQ ID NO: 15 and each of the short chains comprises the sequence of SEQ ID NO: 16; or
(i) each of the long chains comprises the sequence of SEQ ID NO: 17 and each of the short chains comprises the sequence of SEQ ID NO:18; or
(j) each of the long chains comprises the sequence of SEQ ID NO:19 and each of the short chains comprises the sequence of SEQ ID NO:20; or
(k) each of the long chains comprises the sequence of SEQ ID NO:21 and each of the short chains comprises the sequence of SEQ ID NO:22; or
(l) each of the long chains comprises the sequence of SEQ ID NO:23 and each of the short chains comprises the sequence of SEQ ID NO:24.

11. The ADC, or the pharmaceutically acceptable salt thereof, of claim 1, wherein the long chains each comprises a sequence selected from the group consisting of SEQ ID NOs: 3, 5, and 9, and wherein the short chains each comprises a sequence selected from the group consisting of SEQ ID NOs: 4, 6, and 10.

12. The ADC, or the pharmaceutically acceptable salt thereof, of claim 1, wherein the first segment and second segment of each of the long chains each comprises a sequence selected from the group consisting of SEQ ID NOs: 25, 26, 27, 28, 29, 30, 31, 32, 33, and 34.

13. The ADC, or the pharmaceutically acceptable salt thereof, of claim 1, wherein the first segment and second segment of each of the short chains each comprises a sequence selected from the group consisting of SEQ ID NOs: 25, 26, 27, 28, 29, 30, 31, 32, 33, and 34.

14. The ADC, or the pharmaceutically acceptable salt thereof, of claim 1, wherein:
(a) each of the long chains comprises the sequence of SEQ ID NO:25 and each of the short chains comprises the sequence of SEQ ID NO:26; or
(b) each of the long chains comprises the sequence of SEQ ID NO:26 and each of the short chains comprises the sequence of SEQ ID NO:25; or
(c) each of the long chains comprises the sequence of SEQ ID NO:27 and each of the short chains comprises the sequence of SEQ ID NO:28; or
(d) each of the long chains comprises the sequence of SEQ ID NO:28 and each of the short chains comprises the sequence of SEQ ID NO:27; or
(e) each of the long chains comprises the sequence of SEQ ID NO:29 and each of the short chains comprises the sequence of SEQ ID NO:30; or
(f) each of the long chains comprises the sequence of SEQ ID NO:30 and each of the short chains comprises the sequence of SEQ ID NO:29; or
(g) each of the long chains comprises the sequence of SEQ ID NO:31 and each of the short chains comprises the sequence of SEQ ID NO:32; or
(h) each of the long chains comprises the sequence of SEQ ID NO:32 and each of the short chains comprises the sequence of SEQ ID NO:31; or
(i). each of the long chains comprises the sequence of SEQ ID NO:33 and each of the short chains comprises the sequence of SEQ ID NO:34; or
(j) each of the long chains comprises the sequence of SEQ ID NO:34 and each of the short chains comprises the sequence of SEQ ID NO:33.

15. The ADC, or the pharmaceutically acceptable salt thereof, of claim 1, wherein the second segment is connected to the remainder of the long chain via a flexible peptide linker comprising one or more repeat units of the sequence of (GₓS)_{y}, where x is a whole number between 1 and 6 inclusive and y is a whole number between 0 and 8 inclusive.

16. The ADC, or the pharmaceutically acceptable salt thereof, of claim 1, wherein the antigen is selected from the group consisting of EGFR, HER2, and c-MET.

17. The ADC, or the pharmaceutically acceptable salt thereof, of claim 1, wherein each of the short chains comprises a sequence selected from the group consisting of SEQ ID NO:2, SEQ ID NO: 4, SEQ ID NO:6, SEQ ID NO:8, SEQ ID NO: 10, SEQ ID NO: 12, SEQ ID NO: 14, SEQ ID NO: 16, SEQ ID NO:18, SEQ ID NO:20, SEQ ID NO:22, and SEQ ID NO:24.

18. The ADC, or the pharmaceutically acceptable salt thereof, of claim 1, wherein each of the long chains comprises a sequence selected from the group consisting of SEQ ID NO:1, SEQ ID NO: 3, SEQ ID NO:5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO:21, and SEQ ID NO:23.

19. An ADC or a pharmaceutically acceptable salt thereof, comprising:
a monoclonal antibody conjugated to a cytotoxic drug by a chemical linker, wherein the monoclonal antibody comprises two identical long chains and four identical short chains, wherein:
(a) each of the long chains comprises the sequence of SEQ ID NO:1 and each of the short chains comprises the sequence of SEQ ID NO:2; or
(b) each of the long chains comprises the sequence of SEQ ID NO:3 and each of the short chains comprises the sequence of SEQ ID NO:4; or
(c) each of the long chains comprises the sequence of SEQ ID NO:5 and each of the short chains comprises the sequence of SEQ ID NO:6; or
(d) each of the long chains comprises the sequence of SEQ ID NO:7 and each of the short chains comprises the sequence of SEQ ID NO:8; or
(e) each of the long chains comprises the sequence of SEQ ID NO:9 and each of the short chains comprises the sequence of SEQ ID NO: 10; or
(f) each of the long chains comprises the sequence of SEQ ID NO: 11 and each of the short chains comprises the sequence of SEQ ID NO: 12; or
(g) each of the long chains comprises the sequence of SEQ ID NO: 13 and each of the short chains comprises the sequence of SEQ ID NO:14; or
(h) each of the long chains comprises the sequence of SEQ ID NO:15 and each of the short chains comprises the sequence of SEQ ID NO: 16; or
(i) each of the long chains comprises the sequence of SEQ ID NO: 17 and each of the short chains comprises the sequence of SEQ ID NO: 18; or
(j) each of the long chains comprises the sequence of SEQ ID NO: 19 and each of the short chains comprises the sequence of SEQ ID NO:20; or
(k) each of the long chains comprises the sequence of SEQ ID NO:21 and each of the short chains comprises the sequence of SEQ ID NO:22; or
(l) each of the long chains comprises the sequence of SEQ ID NO:23 and each of the short chains comprises the sequence of SEQ ID NO:24.

20. The ADC, or the pharmaceutically acceptable salt thereof, of claim 1, wherein the cytotoxic drug is selected from the group consisting of monomethyl auristatin E (MMAE), monomethyl auristatin F (MMAF), auristatin E, auristatin F, maytansine DM1 and DM4, maytansinol, sandramycin, pyrrolobenzodiazepine dimer, anthracyclines, calicheamicin, dolastatin 10, duocarmycin, doxorubicin, thailanstatin A, uncialamycin, amanitins, ricin, diphtheria toxin, eribulin, ¹³¹I, interleukins, tumor necrosis factors, chemokines, and nanoparticles.

21. The ADC, or the pharmaceutically acceptable salt thereof, of claim 1, wherein the chemical linker comprises a portion which is selected from the group consisting of 6-maleimidocaproyl (MC), maleimidopropionyl (MP), valine-citrulline (val-cit), alanine-phenylalanine (ala-phe), p-aminobenzyloxycarbonyl (PAB), N-succinimidyl 4-(2-pyridylthio) valerate (SPP), N-succinimidyl 4-(N-maleimidomethyl)-cyclohexane-1-carboxylate (SMCC), N-succinimidyl (4-iodo-acetyl) aminobenzoate (SIAB), 6-maleimidocaproyl-valine-citrulline-p-aminobenzyloxycarbonyl (MC-vc-PAB), Val-Cit-PABC, N-succinimidyl-4-(2-pyridyldithio) butanoate (SPDB), N-succinimidyl 3-(pyridin-2-yldithio)-propionate (SPDP), Phe-Lys (Fmoc)-PAB, Aloc-D-Ala-Phe-Lys (Aloc)-PAB-PNP, Boc-Phe-(Alloc) Lys-PAB-PNP, and perfluorophenyl 3-(pyridine-2-yldisulfanyl) propanoate.

22. The ADC, or the pharmaceutically acceptable salt thereof, of claim 21, wherein the chemical linker comprises a PEGn spacer wherein n is a whole number between 1 and 20.

23. The ADC, or the pharmaceutically acceptable salt thereof, of claim 1, wherein the chemical linker further comprises a valine-citrulline dipeptide linker connected to the PEGn spacer.

24. The ADC, or the pharmaceutically acceptable salt thereof, of claim 1, wherein the drug-to-antibody ratio is between 1 and 12 inclusive.
